(19) United States
(12) Patent Application Publication
(10) Pub. No.: US 2007/0015971 A1
(43) Pub. Date: Jan. 18, 2007

(54) Disease predictions

(76) Inventors: Shankara Rao Arvind Atignal, Karnataka (IN); Anuradha Rajput, Kamataka (IN); Halasingana Halli Lingappa Hanume Gowda, Kamataka (IN); Mandyam Krishnakumar Narasimha, Kamataka (IN); Subramaniam Kalyanasundaram, Kamataka (IN); Vijay Chandru, Kamataka (IN)

Correspondence Address: CHOATE, HALL & STEWART LLP, TWO INTERNATIONAL PLACE, BOSTON, MA, 02110, US

(21) Appl. No.: 10/555,225

(22) PCT Filed: May 14, 2003
(86) PCT No.: PCT/IN03/00190

§ 371 Date: Nov. 1, 2005

(51) Int. Cl. A61B 5/00

(52) U.S. Cl. 600/300; 128/920; 128/925

(57) ABSTRACT

A support vector machine (110) is used to predict who, among a population of patients with diabetes mellitus, will develop proteinuria which is in indicator of diabetic nephropathy. The support vector machine (110) is trained using test results of the patients from blood biochemistry and haemotology tests. The training and testing of the support vector machine (110) used data in which the entire patient population did not exhibit signs of proteinuria at a predetermined time period and three months later, and some of the patient population had proteinuria six months from the predetermined time period. The support vector machine (110) is used to predict who, among patients with diabetes mellitus using lest results from a predetermined time period and three months later, will develop proteinuria at six months from the predetermined time period. The input data to the support vector machine (110) included different parameters of test results at a predetermined time and three months later.

### FIELD OF THE INVENTION

This application relates to prediction of complications of disease processes, and more particularly, to selection of concentrated samples of patients who may develop a particular complication from among the patients with a particular disease.

### BACKGROUND OF THE INVENTION

Patients suffering from a disease, such as diabetes mellitus, may run an increased risk of developing certain complications, such as developing diabetic nephropathy. Nephropathy is a complication of diabetes mellitus. Proteinuria is one of the early signs of nephropathy. After the onset of certain complications, such as diabetic nephropathy, a patient's condition may not be improved even with proper treatment. Generally, earlier detection and treatment of a complication results in increased chances of improvement and prognosis for the patient. Thus, it may be desirable to improve diagnosis of conditions, diseases and related complications, such as diabetic nephropathy, as early as possible. It may be desirable to perform such a diagnosis efficiently and accurately prior to the onset of the condition in the patient.

### SUMMARY OF THE INVENTION

In accordance with one aspect of the invention, the limitations of early detection of diabetic nephropathy are overcome by providing a method and tool/system for predicting diabetic nephropathy in individuals suffering from diabetes. One embodiment of the invention identifies a group of six parameters whose function serves as a biomarker to predict whom, among the diabetic patients, will be afflicted with the condition of nephropathy in the future.

In accordance with yet another aspect of the invention is a machine used to predict a certain complication of a certain disease with appropriate choice of test measurements and their functional relationship with the assistance of machine learning techniques.

In accordance with one aspect of the invention is a method of disease prediction. A machine learning tool is used to predict whether a member from a first class will belong to a second class after a predetermined amount of time. Members of the first class and the second class have a particular disease. Members of the first class do not have a particular complication after a predetermined amount of time and members of the second class do have the particular complication after the predetermined amount of time.

In accordance with another aspect of the invention is a computer program product used for disease prediction. Included in the computer program product is a machine learning tool that predicts whether a member from a first class will belong to a second class after a predetermined amount of time. Members of the first class and the second class have a particular disease, and members of the first class do not have a particular complication after the predetermined amount of time and members of the second class do have the particular complication after the predetermined amount of time.

In accordance with yet another aspect of the invention is a method of producing a support vector machine used in disease prediction. An input data set is partitioned into a training data set and a testing data set. The input data set includes members belonging to a first class and members belonging to a second class. Members of the first class and the second class have a particular disease, and members of the first class do not have a particular complication at a first time period and three and six months after the first time period. Members of the second class have the particular complication at six months from the first time period, but not at the first time period and three months later.

In accordance with yet another aspect of the invention is a computer program product that produces a support vector machine used in disease prediction. It includes machine executable code that partitions an input data set into a training data set and a testing data set. The input data set includes members belonging to a first class and members belonging to a second class. Members of the first class and the second class have a particular disease, and members of the first class do not have a particular complication at a first time period and three and six months after the first time period and members of the second class have the particular complication at six months from the first time period, but not at the first time period and three months later.

In accordance with still another aspect of the invention is method of disease prediction. A support vector machine is used to predict whether a member from a first class will belong to a second class after a predetermined amount of time. Members of the first class and the second class have diabetes mellitus, and members of the first class do not have proteinuria after the predetermined amount of time and members of the second class do have proteinuria after the predetermined amount of time. The input data of a patient used to predict whether the patient will belong to the first class or the second class includes input parameters based on test results including potassium, SGPT, glycosylated haemoglobin, cholesterol, chloride and LDL.

In accordance with yet another aspect of the invention is a computer program product used for disease prediction. Included is a support vector machine that predicts whether a member from a first class will belong to a second class after a predetermined amount of time. Members of the first class and the second class have diabetes mellitus, and members of the first class do not have proteinuria after the predetermined amount of time and members of the second class do have proteinuria after the predetermined amount of time. The input data of a patient used to predict whether the patient will belong to the first class or the second class includes input parameters based on test results including potassium, SGPT, glycosylated haemoglobin, cholesterol, chloride and LDL.

In accordance with another aspect of the invention is a computer-implemented method for disease prediction. It is predicted whether a member from a first class will belong to a second class after a predetermined amount of time. Members of the first class and the second class have diabetes mellitus, and members of the first class do not have proteinuria after the predetermined amount of time and members of the second class do have proteinuria after the predetermined amount of time. The input data of a patient used to predict whether the patient will belong to the first class or the second class includes input parameters based on test results including potassium, SGPT, glycosylated haemoglobin, cholesterol, chloride and LDL.

In accordance with another aspect of the invention is a computer program product for disease prediction. Included is machine executable code that predicts whether a member from a first class will belong to a second class after a predetermined amount of time. Members of the first class and the second class have diabetes mellitus, and members of the first class do not have proteinuria after the predetermined amount of time and members of the second class do have proteinuria after the predetermined amount of time. The input data of a patient used to predict whether the patient will belong to the first class or the second class includes input parameters based on test results including potassium, SGPT, glycosylated haemoglobin, cholesterol, chloride and LDL.

In accordance with still another aspect of the invention is a computer-implemented method for producing a machine-learning tool used in disease prediction. The machine-learning tool is trained using training data to predict whether a member from a first class will belong to a second class after a predetermined amount of time. Members of the first class and the second class have diabetes mellitus, and members of the first class do not have proteinuria after the predetermined amount of time and members of the second class do have proteinuria after the predetermined amount of time. The training data includes, for each patient, input parameters based on test results including potassium, SGPT, glycosylated haemoglobin, cholesterol, chloride and LDL.

In accordance with yet another aspect of the invention is a computer program product for producing a machine-learning tool used in disease prediction. Included is machine executable code that trains the machine-learning tool using training data to predict whether a member from a first class will belong to a second class after a predetermined amount of time. Members of the first class and the second class have diabetes mellitus, and members of the first class do not have proteinuria after the predetermined amount of time and members of the second class do have proteinuria after the predetermined amount of time. The training data includes, for each patient, input parameters based on test results including potassium, SGPT, glycosylated haemoglobin, cholesterol, chloride and LDL.

### BRIEF DESCRIPTION OF THE DRAWINGS

Features and advantages of the present invention will become more apparent from the following detailed description of exemplary embodiments thereof taken in conjunction with the accompanying drawings in which:

FIG. 1 is an example of an embodiment of a computer system according to the present invention;

FIG. 2 is an example of an embodiment of a data storage system of the computer system of FIG. 1;

FIG. 3 is an example of an embodiment of components that may be included in a host system of the computer system of FIG. 1;

FIG. 4 is an example of an embodiment of data flow for a support vector machine (SVM);

FIG. 5 is an illustration of a linear separating surface separating input data into two classes with representative support vectors;

FIG. 6 is an illustration of a non-linear separating surface separating input data into two classes with representative support vectors;

FIG. 7 is a flowchart of steps of one embodiment for training, validating and using a support vector machine for classifying data; and

FIG. 8 is a flowchart of method steps of one embodiment for performing training and validation of a support vector machine (SVM).

### DETAILED DESCRIPTION OF THE INVENTION

Referring now to FIG. 1, shown is an example of an embodiment of a computer system that may be used with the techniques described herein. The computer system 10 includes a data storage system 12 connected to host systems 14a-14n through communication medium 18. In this embodiment of the computer system 10, the N hosts 14a-14n may access the data storage system 12, for example, in performing input/output (I/O) operations or data requests. The communication medium 18 may be any one of a variety of networks or other type of communication connections as known to those skilled in the art. The communication medium 18 may be a network connection, bus, and/or other type of data link, such as a hardwire, wireless, or other connection known in the art. For example, the communication medium 18 may be the Internet, an intranet, network or other connection(s) by which the host systems 14a-14n may access and communicate with the data storage system 12, and may also communicate with others 15, included in the computer system 10.

Each of the host systems 14a-14n and the data storage system 12 included in the computer system 10 may be connected to the communication medium 18 by any one of a variety of connections as may be provided and supported in accordance with the type of communication medium 18. Each of the processors included in the host computer systems 14a-14n may be any one of a variety of commercially available single or multi-processor system, such as an Intel-based processor, IBM mainframe or other type of commercially available processor able to support incoming traffic in accordance with each particular embodiment and application.

It should be noted that the particulars of the hardware and software included in each of the host systems 14a-14n, as well as those components that may be included in the data storage system 12, are described herein in more detail, and may vary with each particular embodiment. Each of the host computers 14a-14n may all be located at the same physical site, or, alternatively, may also be located in different physical locations. Examples of the communication medium that may be used to provide the different types of connections between the host computer systems and the data storage system of the computer system 10 may use a variety of different communication protocols such as SCSI, ESCON, Fibre Channel, or GIGE (Gigabit Ethernet), and the like. Some or all of the connections by which the hosts and data storage system 12 may be connected to the communication medium 18 may pass through other communication devices, such as a Connectrix or other switching equipment that may exist such as a phone line, a repeater, a multiplexer or even a satellite.

Each of the host computer systems may perform different types of data operations in accordance with different types of tasks. In the embodiment of FIG. 1, any one of the host computers 14a-14n may issue a data request to the data storage system 12 to perform a data operation, such as a read or a write operation.

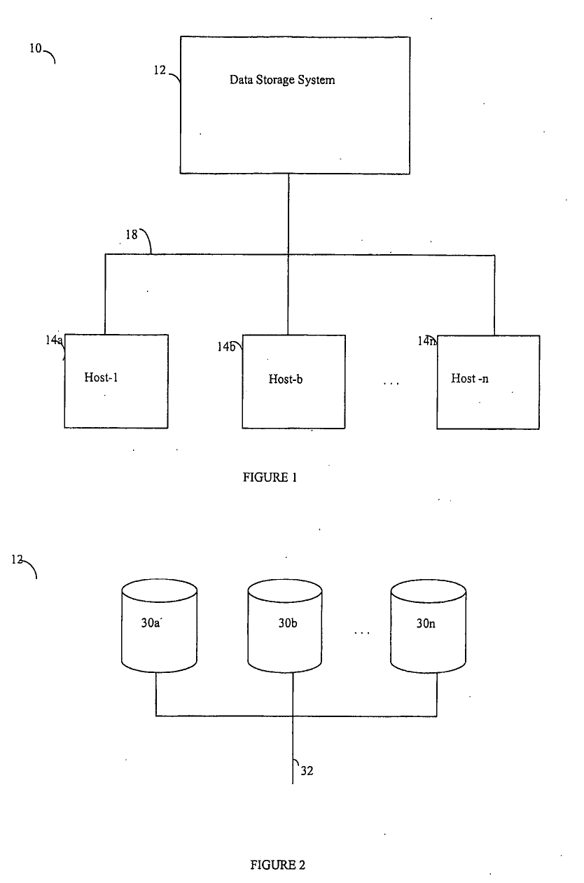

Referring now to FIG. 2, shown is an example of an embodiment of a data storage system 12 that may be included in the computer system 10 of FIG. 1. The data storage system 12 in this example may include a plurality of data storage devices 30a through 30n. The data storage devices 30a through 30n may communicate with components external to the data storage system 12 using communication medium 32. Each of the data storage devices may be accessible to the hosts 14a through 14n using an interface connection between the communication medium 18 previously described in connection with the computer system 10 and the communication medium 32. It should be noted that a communication medium 32 may be any one of a variety of different types of connections and interfaces used to facilitate communication between communication medium 18 and each of the data storage devices 30a through 30n.

The data storage system 12 may include any number and type of data storage devices. For example, the data storage system may include a single device, such as a disk drive, as well as a plurality of devices in a more complex configuration, such as with a storage area network and the like. Data may be stored, for example, on magnetic, optical, or silicon-based media. The particular arrangement and configuration of a data storage system may vary in accordance with the parameters and requirements associated with each embodiment.

Each of the data storage devices 30a through 30n may be characterized as a resource included in an embodiment of the computer system 10 to provide storage services for the host computer systems 14a through 14n. The devices 30a through 30n may be accessed using any one of a variety of different techniques. In one embodiment, the host systems may access the data storage devices 30a through 30n using logical device names or logical volumes. The logical volumes may or may not correspond to the actual data storage devices. For example, one or more logical volumes may reside on a single physical data storage device such as 30a. Data in a single data storage device may be accessed by one or more hosts allowing the hosts to share data residing therein.

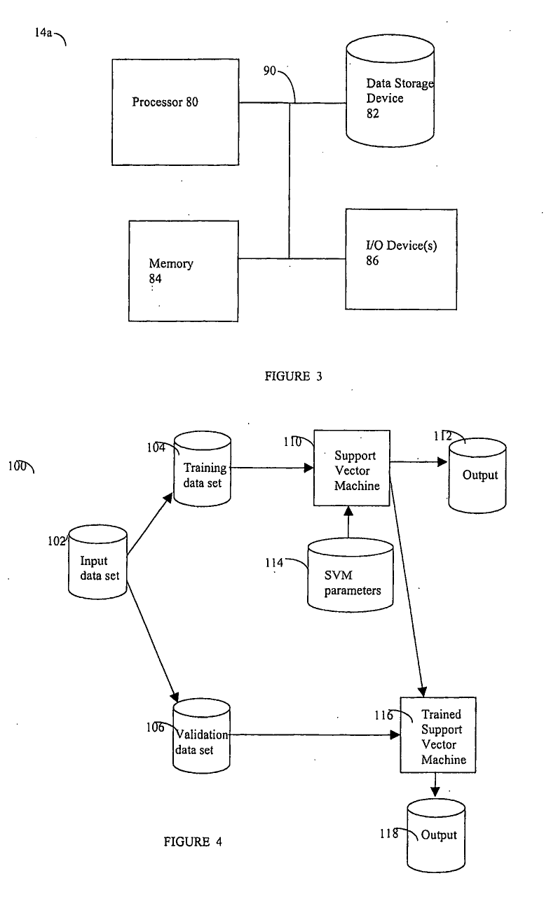

Referring now to FIG. 3, shown is an example of an embodiment of a host or user system 14a It should be noted that although a particular configuration of a host system is described herein, other host systems 14b-14n may also be similarly configured. Additionally, it should be noted that each host system 14a-14n may have any one of a variety of different configurations including different hardware and/or software components. Included in this embodiment of the host system 14a is a processor 80, a memory, 84, one or more I/O devices 86 and one or more data storage devices 82 that may be accessed locally within the particular host system. Each of the foregoing may communicate using a bus or other communication medium 90. Each of the foregoing components may be any one of more of a variety of different types in accordance with the particular host system 14a.

Computer instructions may be executed by the processor 80 to perform a variety of different operations. As known in the art, executable code may be produced, for example, using a loader, a linker, a language processor, and other tools that may vary in accordance with each embodiment. Computer instructions and data may also be stored on a data storage device 82, ROM, or other form of media or storage. The instructions may be loaded into memory 84 and executed by processor 80 to perform a particular task. One embodiment uses a Java-based programming language to implement the techniques described herein on a LINUX operating system running on any one of a variety of commercially available processors, such as may be included in a personal computer.

Referring now to FIG. 4, shown is an example of an embodiment of components that may be included in a support vector machine (SVM) classifier system 100. The example 100 shows data flow between the components. The components of the SVM classifier system 100 may reside and be executed on one or more of the host computer systems included in the computer system 10 of FIG. 1. The SVM is one type of machine learning tool that may be used in connection with disease prediction and prediction of complications associated with a disease. This is described in more detail in following paragraphs. One embodiment of an SVM, like other machine learning tools, operates in two phases: a training phase and a testing or validation phase. The system 100 includes an input data set 102 that is partitioned into a training data set 104 and a validation data set 106 each used, respectively, in the training and validation phases. SVMs and other types of machine learning tools and techniques are described, for example, in Nello Cristianini and John Shawe-Taylor: An introduction to Support Vector Machines, Cambridge University Press, 2000, and in V. Vapnik, Statistical learning theory, Weily, 1998.

The training data set 104 may be used as input to the SVM 110 in the training phase. SVM parameters 114 may also be selected as initial inputs to the SVM 110. It should be noted that the SVM parameters 114 may be adjusted and tuned in accordance with predetermined criteria. The SVM 110 produces output 112 during its training. Subsequently, the trained SVM 116 is produced as a result of the training phase and is tested using the validation data set 106. If the output 118 produced by the trained SVM 116 meets predetermined criteria, the trained SVM 116 may be used as a classifier for other input data. Otherwise, adjustments may be made such that the resulting trained SVM 116 classifies input data in accordance with predetermined criteria. Adjustments may include, for example, modification to the SVM parameters, using different features based on the training data set, and the like.

Generally, in connection with an SVM, an object or element to be classified may be represented by a number of the features. If, for example, the object to be classified may be represented by two features, the object may be represented by a point of two dimensional spaces. Similarly, if the object to be classified may be represented by N features, also referred to as a feature vector, the object may be represented by a point in N dimensional space. An SVM defines a plane in the N dimensional space which may also be referred to as a hyperplane. This hyperplane separates feature vector points associated with objects in a particular class and feature vector points associated with objects not in a defined class.

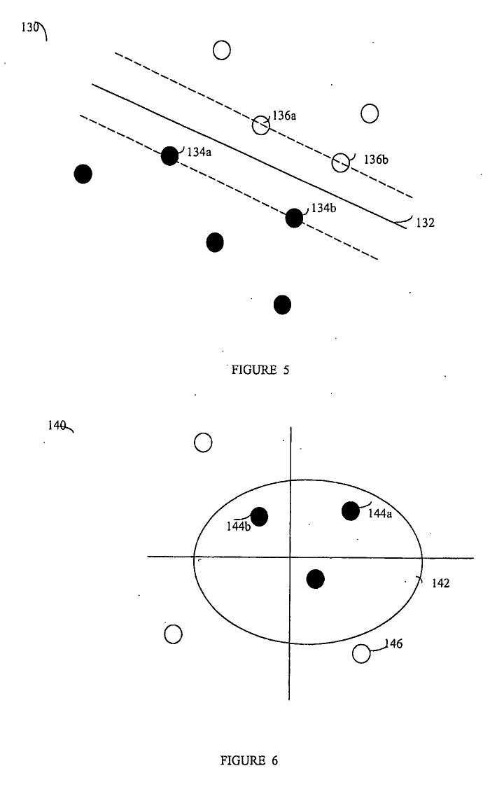

For example, referring now to FIG. 5, shown is an illustration 130 representing how a linear separating surface separates feature vector points. In the illustration 130, the plane or surface 132 may be used to separate feature vector points denoted with blackened circles associated with objects in the class. These blackened circles may be separated by the hyperplane 132 from other objects denoted as not belonging to the class. Objects not in the class are denoted as having hollow circles. A number of hyperplanes may be defined to seperate any given pair of classes. Training an SVM involves defining a hyperplane that has maximal distance, such as the Euclidian distance, from the hyperplane to the closest point or points. These closest point or points may also be referred to as support vectors. The hyperplane maximizes the Euclidian distance, for example, between points in the class and points not in the class. Referring back to FIG. 5, example support vectors in this illustration are denoted as 134a, 134b, 136a and 136b.

An SVM as described herein may be characterized as a two-class classifier having a decision rule which takes the general form:

$$Y = {\sum }_{i = 1}^{Ns} {α}_{i} K ( x , {s}_{i} ) {m}_{i} + b$$

where sᵢ, Ns, b, mᵢ and αᵢ are parameters of the SVM and x is the vector to be classified. The SVM training process determines sᵢ, Ns, b and αᵢ. The resulting sᵢ's, i=1, . . . , Ns are a subset of the training set referred to as support vectors.

Referring back to FIG. 5, the decision function represented is a linear function of the data. There are instances in which a decision function is not a linear function of the data. In other words, the separating surface separating the classes is not linear.

Referring now to FIG. 6, shown is an illustration 140 of a non-linear separating surface which separates feature vector points. In the illustration 140, the curve 142 separates feature vector points included in a first class, as denoted with blackened circles, from other feature vector points not included in the first class, as denoted with hollow circles. Points 144a, 144b and 146 may be referred to as example support vectors. In connection with nonlinear SVMs, a kernel function may also be used in defining the decision rule.

Choice of a particular kernel function determines whether the resulting SVM is a nomial or Gaussian classifier. As described above, a decision rule for an SVM is a function of the corresponding kernel function and support vectors. A data point in one embodiment, as described in more detail elsewhere herein, represents characteristics about a patient. The data point may be represented as a vector that has one or more coordinates. The SVM is trained using the training dataset. Subsequently, the testing or validation dataset may be used after training to make a determination as to whether a particular configuration of the SVM provides an optimal solution.

An SVM, which is one particular type of a learning machine may be trained, for example, by adjusting operating parameters until a desirable training output is achieved. A determination of whether a training output is desirable may be accomplished, for example, by manual detection and determination, and/or by automatically comparing training output to known characteristics of training data. A learning machine may be considered to be trained when its training data is within a predetermined error threshold from the known characteristics of the actual training data. The predetermined error threshold or criteria may vary in accordance with each embodiment.

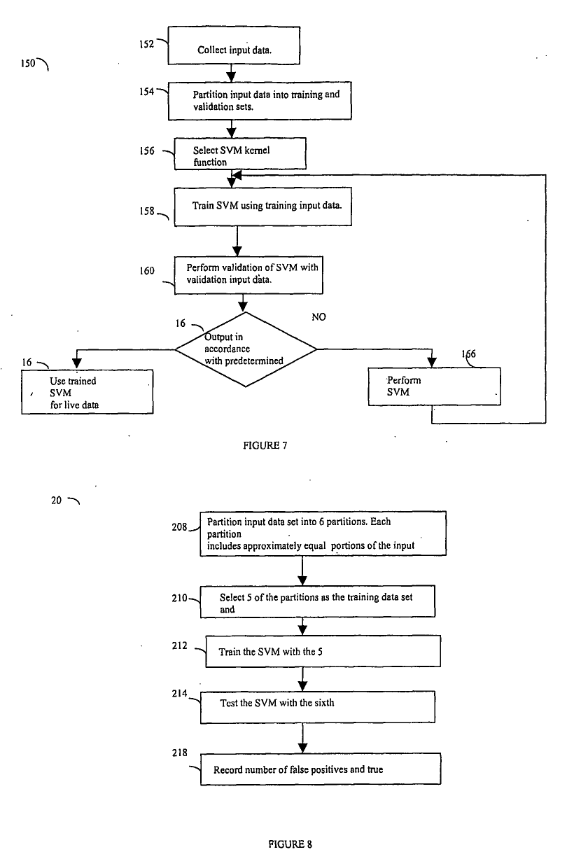

Referring now to FIG. 7, shown is a flowchart 150 of steps of one embodiment for producing a trained SVM used for data classification. At step 152, the problem is determined and input data is collected. At step 154, the input data is partitioned into training and validation data sets. Subsequently, in connection with use of an SVM in this embodiment, an SVM kernel function and associated parameters are selected. Kernels may be selected for use in connection with an SVM in accordance with any one of a variety of different types of criteria. A kernel function may be selected based on prior performance knowledge. For example, exemplary kernels include polynomial kernels, Gaussian kernels, linear kernels, and the like. An embodiment may also select and utilize a customized kernel that may be created specific to a particular problem or type of dataset. Kernel functions as used in SVMs are described, for example, in Nello Cristianini and John Shawe-Taylor: An introduction to Support Vector Machines, Cambridge University Press, 2000.

At step 158, the SVM is trained using the training data set. It should be noted that an embodiment may also include an optional preprocessing step to pre-process the input data set to determine the difference parameters described in following paragraphs. Other embodiments may include other pre-processing steps. At step 160, the trained SVM is validated or tested using the validation input data. At step 162, the output of the trained SVM is examined and a determination is made as to whether the output produced by the trained SVM is in accordance with the predetermined criteria, such as an acceptable level or error threshold. This may vary with each embodiment. In one embodiment, the predetermined criteria includes a specified number of false positives and/or false negatives. If the output of the trained SVM does not meet the one or more predetermined criteria, control proceeds from step 162 to step 166 where SVM adjustments may be made. In one embodiment, this may include selection of different kernel functions and/or parameters. Control proceeds to step 158 where the training and validation steps are repeated until the trained SVM classifies data in accordance with the predetermined output. Once the SVM is trained and classifies input data in accordance with the predetermined criteria, control proceeds to step 164 where the trained SVM may be used for live data classification.

As described in more detail elsewhere herein, in one embodiment, a machine learning predicting tool, such as the SVM, may be used to predict with a specified degree of accuracy as the predetermined criteria whether a patient develops a particular condition, such as diabetic nephropathy, a complication of the disease diabetes mellitus, at least three months in advance.

In one embodiment, the inputs to the SVM are a subset of routine laboratory measurements which are the results of tests performed using the blood and urine samples from patients. A trained machine learning predicting tool may use the numerical values of these test results to predict whether a diabetic patient will develop diabetic nephropathy, for example, in the subsequent three months.

It should be noted that the test results used as an input to the SVM as described herein are not used currently by the medical profession for either the diagnosis or the prediction of early diabetic nephropathy. Currently, the test results may be used as indicators of some other complications, such as electrolyte imbalance caused by renal failure in nephropathic patients. However, individually or in any combination, these test results have not been demonstrated to be capable of indicating the onset of diabetic nephropathy. As described herein, the machine learning predicting tool may be utilized to find a combination of these test parameters and their functional relationship in order to predict early diabetic nephropathy.

Use of the machine learning predicting tool described herein involves an intelligent way of training a machine to learn from known instances of diabetic nephropathy in a diabetic population. These known instances are used to train the SVM which may then be used as a predictive tool. It should be understood that the techniques described herein are not limited to diabetes mellitus and its complication diabetic nephropathy. Rather, these techniques may be used in connection with predicting other conditions and/or complications associated with other diseases.

As described herein, techniques may be used to train machine learning predicting tools to learn the pattern of disease evolution. With appropriate choice of tests, test results, and functions relating them, predictions may be made with respect to a complication that may develop over time as a result of a diseased condition. It should also be noted that although a particular type of machine learning tool, the SVM, is described herein, the techniques utilized in connection with the SVM may also be used with other diagnostic methods and systems, such as, for example, decision trees, neural networks, cluster analysis, and the like.

In connection with a diabetic population over time, it may be observed that a small fraction of patients typically develop proteinuria for the first time every three months. One embodiment of a machine learning predicting tool may be used to predict who among the patients with diabetes mellitus will develop proteinuria. As described herein, one embodiment may base such predictions using combinations of routine blood biochemistry and haematology test parameters. In order to make such predictions, a portion of the a given set of routine, blood biochemistry and haematology test parameters may be determined. The prediction involves training an SVM.

In one embodiment, the SVM is trained using the input data of difference parameters, described in more detail elsewhere herein, for classification of patients into two classes. In this embodiment, the predetermined criteria used in training the SVM, such as in connection with step 162, are:

the trained SVM should minimize the number of patients falsely identified as developing proteinuria (minimize false positives); and

the trained SVM should maximize the number of patients correctly identified as developing proteinuria (maximize true positives).

An SVM, when trained with an appropriate choice of a subset of difference parameters and an appropriate choice of the internal SVM parameters, may achieve the above-mentioned two goals of minimizing the false positives and maximizing the true positives. An embodiment may specify limits or thresholds with one or both of the foregoing.

In connection with training the SVM, one embodiment uses the input data of the blood biochemistry and haematology test reports of 187 diabetic patients who were tested once within each of three three-month time periods. In other words, a set of input data is associated with each of 187 patient's test reports for time periods 0, 3, and 6 months. Input data sets associated with each of the time periods 0, 3 and 6-months are referred to herein, respectively, as Trials 1, 2, and 3. The same set of the blood biochemistry and haematology tests were carried out in each of the Trials 1, 2 and 3 for all the 187 patients. The test results indicated that none of the patients showed proteinuria in the first two trials. Only twelve (12) of the 187 patients showed proteinurea in the third Trial. All the twelve patients who developed proteinurea in the third Trial are classified as class 2 patients and the remainder of the 187 patients are classified as class 1 patients.

The blood biochemistry tests performed were albumin, alkaline phosphates, SGOT, SGPT, calcium, cholesterol, chloride, creatinine kinase, creatinine, bicarbonate, iron, gamma GT, glucose, HDL cholesterol, potassium, lactate dehydrogenase, LDL, magnesium, sodium, phosphorus, total bilirubin, total protein, triglycerides, UIBC, urea, uric acid, glycosylated haemoglobin.

The urinalysis tests performed were pH, specific gravity, glucose, protein, ketones, urobilinogen, bilirubin, nitrites, leukocytes, erythrocytes, epithelial cells, casts, crystals.

The haematology tests performed were white blood cells, differential counts, neutrophils, lymphocytes, monocytes, eosinophils, basophils, red blood corpuscles, hemoglobin, hematocrit, mean cell volume, mean cell hemoglobin, mean cell haemoglobin concentration, platelet count, erythrocyte sedimentation rate, reticulocyte count, peripheral smear, and blood grouping.

The selection of which of the foregoing test results to use in one embodiment, and the difference parameters thereof, were made using feature selection tools, such as analysis of varience, Kruskal-Wallis Test and matrix plots as well as intuitive prediction based upon empirical knowledge from several such experiments. The foregoing feature selection tools and techniques, as well as others that may be used in an embodiment, are known in the art and described, for example, in Stanton a. Glantz: Primer of Biostatistics,

McGraw-Hill, 2002.

One embodiment trains an SVM using the knowledge of the blood biochemistry and haematology tests of the 187 patients. Subsequently, the trained SVM may be used in to identify a patient as belonging to class 1 or class 2. The blood biochemistry and haematology test reports of a new diabetic patient who did not have proteinurea up to the current time period are given as input to the trained SVM. The test reports are for time periods of 0 months and 3 months. The trained SVM determines whether the new patient will belong to class 1 or class 2 for the next time period which, in this embodiment is whether the patient's test results will indicate proteinurea three months later (time=6 months with respect to the first test report at time 0.

In one embodiment, input data is prepared using the clinical data consisting of the 45 blood biochemistry and haematology tests, as set forth above, for a population of 187 patients repeated at time 0 and time 3 months.

The 45 tests done at time 0 months are denoted by

b(0,j,1),b(0,j,2), . . . ,b(0,j,45)
   The 45 tests done at time 3 months are denoted by
b(3,j,1),b(3,j,2), . . . ,b(3,j,45)
   The difference of the foregoing at two times, such as at time 0 and 3 months later, is represented as follows:
d(j,k)=b(0,j,k)−b(3,j,k) for each patient j and each test k.

For each test k for all of the 187 patients, the set {d (1,k), d (2,k), d (3,k), ??, d(187,k)} of differences define a new parameter called the difference parameter.

One embodiment uses the foregoing to determine 45 difference parameters for each of the 45 tests for all the 187 patients.

In one embodiment, one or more of the foregoing 45 difference parameters may be selected for use in training the SVM. In particular, a subset ‘S’ of the 45 difference parameters is selected in one embodiment for use in training the SVM. The subset ‘S’ has ‘p’ elements or difference parameters. For each patient j and each test k that belongs to the subset S, the numerical value d(j,k) may be obtained by a difference in test results of the test k at time 0 and 3 months for patient j. Thus, p such values are generated for each patient such that each of the p number of values of the difference parameters in S may be represented as a p-dimensional vector. Specific examples are given elsewhere herein.

Processing steps performed by an embodiment of the SVM are described in following paragraphs. In one embodiment, the SVM identifies each patient by a unique point in a p-dimensional space whose coordinates are defined by the vector described above. In the embodiment described in this example, there are 187 points in a p-dimensional space, one point for each patient.

The SVM in this embodiment is also supplied with the class labels indicating whether a point, or patient, belongs to class 1 (−1) or to class 2 (+1). The SVM separates the points in this p-dimensional space into class 1 and class 2 by a (p−1)-dimensional separating surface.

The subset of the 187 input points that define this surface are called the support vectors. As known in the art of SVMs, the separating surface can be either linear or non-linear. In the embodiment described herein, the separating surface is non-linear. The non-linearity of such separating surface allows the SVM to separate out intertwined sets of points which, in this embodiment, correspond to patients. The particular type of separating surface and other SVM parameters may vary in accordance with each embodiment, data sets, and/or application.

In this embodiment, part of the training process for the SVM includes finding the kernel function which maps (transforms) each of the support vector points into a different p-dimensional space where the separating surface is linear.

Let Sₙ={d(n,1),d(n,2),d(n,3), . . . ,d(n,p)} denote the vector of difference parameters for the patient number n, (1≦n≦187)). The Gaussian kernel function for the p difference parameters is given by
K(x, sₙ)=e^{M}
and

$$M = {\sum }_{1}^{p} {( {x}_{i} - d ( n , i ) )}^{2} / σ .$$

in which Sn denotes the support vector, xᵢ is a point to be classified, σ is a user settable parameter determined in the training phase. It should be noted that Gaussian kernel functions are described, for example, in Nello Cristianini and John Shawe-Taylor: An introduction to Support Vector Machines, Cambridge University Press, 2000. The above-referenced Gaussian kernel function has been defined for use in this embodiment to include the difference parameters as described herein.

In one embodiment, training the SVM includes determining and using the following:

(i) one or more blood biochemistry and haematology

parameters, referred herein as ‘set B’.
   (ii) one or more of the internal SVM parameters, referred
herein as ‘set I’.

In this embodiment, as also described elsewhere herein, the guidelines for selecting the one or more members of set B and set 1 include as predetermined criteria minimizing false positives and maximizing true positives, in that order of priority. In one embodiment, particular combinations of members for set I and/or set B may be ranked in accordance with the predetermined criteria such that if a first combination produces no false positives, this first combination may be preferred over a second combination producing one or more false positives. In connection with step 162, for example, described in flowchart 150, an embodiment may continue training until a particular selection of SVM parameters and blood biochemistry and haemotology parameters results in no false positives. Other embodiments may use different criteria in determining an optimal SVM and/or features of the input data.

As described elsewhere herein, in one embodiment, there are two classes of diabetic patients: class 1 patients that do not develop proteinurea in all the three trials at times 0, 3 and 6 months, and class 2 patients that develop proteinurea in the third trial, that is at time 6 months. What will now be described are processing steps in this one embodiment using the foregoing collected input data with an SVM.

Referring now to FIG. 8, shown is a flowchart 200 of steps of an embodiment for training and testing an SVM. At step 208, the input data set is partitioned into six partitions each including approximately the same number of patients. In this embodiment, each partition includes exactly two patients who are known to belong to class 2. Recall that in data collected described elsewhere herein, twelve of the 187 patients were in class 2. The two class 2 patients associated with each partition may be randomly selected from all the class 2 patients. At step 210, 5 of the partitions are selected as the training data set and a sixth remaining partition is used as the testing data set. At step 212, the SVM is trained with the 5 partitions and then tested at step 214 with the sixth partition. At step 218, the number of false positives and true positives are recorded. The recorded number of true and false positives may be used in evaluating a particular set of SVM parameters and/or features for each patient.

Using the foregoing processing steps, the SVM is trained with five of the six partitions and the trained SVM is tested with the sixth partition. In one embodiment, the steps of flowchart 200 are repeated six times for one complete cycle. In this embodiment, a different partition is tested or designated as the sixth partition in step 210 with each of the six iterations included in each complete cycle. In one embodiment, there are 1000 cycles performed on the data set and the total number of true and false positives for these 1000 cycles are noted. Other embodiments may use different values, such as for the number of partitions, number of cycles, and the like than as used herein.

In one embodiment, a portion of the 45 difference parameters or features is utilized to reduce the dimensionality of the data. Different techniques may be used in determining which parameters to use. An embodiment may use any one or more known techniques with the foregoing difference parameters to identify which difference parameters provide the best class separation for separating class 1 and class 2. One embodiment utilizes statistical tests, such as, for example, the analysis of variance (ANOVA), the Kruskal-Wallis Test, and matrix plots (see Stanton a. Glantz: Primer of Biostatistics, McGraw-Hill, 2002) to determine which of the difference parameters show significant variation across class 1 and class 2. The results of these tests were expressed as P-values for each difference parameter. It may be noted that P-value is defined as the probability of being wrong when asserting that a true difference exists. This is described, for example, in Stanton a. Glantz: Primer of Biostatistics, McGraw-Hill, 2002. In one embodiment described in following paragraphs, for example, the top best difference parameters according to their P-values were chosen.

An embodiment may also use a Matrix plot between any pair of difference parameters. Using Matrix Plots, separability of classes across difference parameters may be inferred. Also, the axes along which the two classes are best separated can be chosen from Matrix Plots for further analysis.

These, and other techniques such as Kruskal-Wallis Test (see Stanton a. Glantz: Primer of Biostatistics, McGraw-Hill, 2002) are known in the art in feature selection.

The SVM as described herein may be used as a predictive tool to determine if a new patient belongs to class 1 or class 2. The new patient N has Z number of blood biochemistry and haematology parameters at time 0 and 3 months. “Z” represents the difference parameters selected, such as the different combination of parameters selected in four examples described in following paragraphs. The trained SVM may be used to determine whether the new patient N belongs to class 1 or 2 at time 6 months.

The Z difference parameters for patient N may be represented as d(N,i), i=1,2, . . . ,Z.

x_{N} represents the vector defining the point for patient N to be classified using the SVM and may be noted as:

x_{N}={d(N,1),d(N,2),d(N,3),d(N,4),d(N,5),? . . . ,d(N,Z)}.
   Whether the patient belongs to class 1 or class 2 may be found by applying the following function to x_{N} in which:

k=the number of support vectors;

αₙ is the Lagrange parameter for the n^{th} patient;

yₙ is the class label for the n^{th} patient which is +1 if in the class 2 and −1 otherwise;

K(x_{N},sₙ) is the kernel function for the N^{th} patient; and

b is the offset.

Techniques for determining values in connection with the above, for example, such as the Lagrange values and the offset values as a result of the training phase, are

$$f ( {x}_{N} ) = {\sum }_{n = 1}^{k} {α}_{n} K ( {x}_{N} , {s}_{n} ) {y}_{n} + {b}_{1}$$

parameters that are computed by standard methods, for example, as explained in V. Vapnik, Statistical Learning Theory, Wiley, 1998.
The foregoing kernel function for the Nth patient, referenced above as K(x_{N},sₙ), may be defined as:
K(x_{N}, sₙ)=e^{M}
in which

$$M = {\sum }_{1}^{z} {( d ( N , i ) - d ( n , i ) )}^{2} / {σ}_{21}$$

where,
d(N,i),i=1,2, . . . ,Z are the values of the difference parameters for patient N, and “n” are the difference parameters of each support vector.
   If f(x_{N})>0 then this patient belong to class 2, otherwise to class 1.

What will now be described are four examples of various combinations of difference parameters and SVM parameters that may be selected for use with the SVM and techniques described herein. As described herein, the fourth and last example may be determined as the “best” in accordance with the predetermined criteria of the number of false positives as described elsewhere herein in more detail. For each of the following four examples, the steps of flowchart 200 were executed for 1000 cycles for each selection of parameters.
In one example SVM embodiment, the four differences parameters: potassium, SGPT, glycosylated haemoglobin and cholesterol were selected. These parameters were chosen using ANOVA, matrix plots and intuition.

The following internal SVM parameters were produced as a result of the SVM training and validation executing the processing steps of flowchart 200 of FIG. 8 using the foregoing 4 difference parameters for the collected input data for the 187 patients:

| Kernel Type | gaussian |
|---|---|
| Sigma | 5.0 |
| Offset | −0.862875 |
| Number of support vectors | 165 |

The following first table includes the difference parameters of the support vectors determined in this embodiment. In the first table, there is one support vector in each row. Each row of data includes a corresponding patient identifier (PT ID) in the first column, the Lagrange multiplier in the second column, class labels (CL) in the third column, and the four difference parameters in the next four columns. Class labels have a value of −1 if the patient does not belong to class 2 and a value of +1 if the patient belongs to class 2. A +1 in the CL column indicates that, at time=6 months, this patient developed proteinuria. Each of the difference parameters in the last four columns of the table represent the difference in the corresponding test results for that parameter between times 0 and 3 months.

| | | | | Alt | | |
|---|---|---|---|---|---|---|
| PT-ID | Lagranges | CL | K | (SGPT) | HBA1C | Chol |
| 0 | 0.61505 | −1 | −0.0999999 | 2 | −2.51 | −32 |
| 1 | 0.130881 | −1 | −0.5 | −3 | −3.4 | −9 |
| 2 | 0.128332 | −1 | −0.4 | −7 | 0.49 | 25 |
| 3 | 0.133546 | −1 | −0.8 | −7 | 0.54 | −47 |
| 4 | 0.0598387 | −1 | −0.3 | −2 | −0.34 | −22 |
| 5 | 0.100124 | −1 | −0.5 | −1 | 0.59 | −4 |
| 6 | 0.0740572 | −1 | −0.4 | −2 | −0.91 | −24 |
| 7 | 1.85798 | −1 | 0.0999999 | 14 | −0.0599995 | 4 |
| 8 | 0.13496 | −1 | 0.3 | −9 | −1.15 | −33 |
| 11 | 0.135492 | −1 | 0.3 | −55 | 0.62 | 12 |
| 12 | 0.0253544 | −1 | −0.5 | −1 | 0.23 | −1 |
| 13 | 0.116461 | −1 | −0.4 | 2 | 0.47 | −21 |
| 14 | 0.120251 | −1 | 0.8 | −6 | −0.87 | 39 |
| 15 | 0.0915704 | −1 | −0.2 | 14 | 1.67 | 23 |
| 16 | 0.0815211 | −1 | 0.4 | 5 | −0.66 | 2 |
| 17 | 0.101647 | −1 | 0 | 1 | −1.96 | 2 |
| 18 | 0.138721 | −1 | 0.5 | 4 | −1.3 | 93 |
| 19 | 0.13993 | −1 | −0.3 | 8 | −0.54 | 4 |
| 20 | 0.0892847 | −1 | 0 | −7 | 0.14 | 36 |
| 21 | 0.0968516 | −1 | 0.3 | 5 | −2.81 | 3 |
| 22 | 0.0789543 | −1 | −0.0999999 | 0 | −0.8 | 6 |
| 23 | 0.111783 | −1 | −0.2 | 1 | −0.23 | 37 |
| 25 | 0.126635 | −1 | 0.1 | −1 | 0.6 | 26 |
| 26 | 0.10789 | −1 | 0 | −8 | 0.59 | 13 |
| 27 | 0.0875873 | −1 | 0.3 | 1 | −2.41 | −6 |
| 28 | 0.138416 | −1 | −0.3 | −1 | −6.33 | 120 |
| 29 | 0.132801 | −1 | 0 | 15 | 1.68 | 14 |
| 30 | 0.0824014 | −1 | 0.2 | 0 | −1.98 | −1 |
| 31 | 0.133027 | −1 | 0.2 | 7 | −2.58 | 62 |
| 32 | 0.104445 | −1 | −0.4 | −6 | −1.47 | 5 |
| 33 | 0.0582661 | −1 | 0 | −1 | −0.33 | 9 |
| 34 | 0.0345833 | −1 | 0 | −3 | 1.45 | 6 |
| 35 | 0.230877 | −1 | −0.1 | −2 | 0.15 | 13 |
| 37 | 0.0444765 | −1 | 0.2 | 2 | 0.84 | −7 |
| 38 | 0.140552 | −1 | −0.0999999 | −9 | 2.13 | 1 |
| 39 | 0.085656 | −1 | 0.0999999 | 0 | −0.61 | −18 |
| 40 | 0.134637 | −1 | 0.2 | −7 | 2.11 | −36 |
| 41 | 0.136216 | −1 | 0.6 | 1 | −3.8 | 24 |
| 42 | 0.133777 | −1 | −0.3 | −19 | −1.78 | −4 |
| 43 | 0.129645 | −1 | 0.3 | −4 | −2.91 | 46 |
| 44 | 0.202729 | −1 | 0.0999999 | −1 | −1.47 | −33 |
| 45 | 0.130308 | −1 | −0.1 | 8 | −1.17 | −50 |
| 46 | 0.130575 | −1 | −0.5 | 4 | −3.57 | 31 |
| 47 | 0.23896 | −1 | 0.5 | 2 | −1.1 | 14 |
| 48 | 0.101275 | −1 | 0.8 | −8 | −2.59 | 11 |
| 50 | 0.137655 | −1 | 0 | 0 | 2.61 | 128 |
| 51 | 0.131247 | −1 | 0.2 | −14 | −0.2 | 2 |
| 52 | 1.00613 | −1 | 0.2 | −1 | 1.45 | 18 |
| 53 | 0.136879 | −1 | −0.5 | −19 | −0.11 | 44 |
| 54 | 0.0230675 | −1 | 0.5 | −2 | 0.25 | −23 |
| 56 | 0.0862423 | −1 | 0.0999999 | −5 | 0.42 | −13 |
| 57 | 0.0762971 | −1 | −0.0999999 | −3 | 0.95 | −12 |
| 58 | 0.133586 | −1 | −0.5 | 3 | 1.98 | 26 |
| 59 | 0.0796157 | −1 | 0.4 | −1 | 0.98 | 2 |
| 60 | 0.0828568 | −1 | 0 | 13 | 0.61 | 24 |
| 61 | 0.137225 | −1 | −0.3 | 9 | 0.31 | 53 |
| 62 | 0.109952 | −1 | 0.6 | 1 | 2.52 | 12 |
| 64 | 0.11511 | −1 | 0.2 | −1 | 0.0600004 | 61 |
| 65 | 0.120444 | −1 | 0.2 | 4 | −1.44 | −13 |
| 68 | 0.0332447 | −1 | −0.5 | −2 | 1.4 | −9 |
| 70 | 0.134817 | −1 | −0.6 | 1 | 1.48 | 47 |
| 71 | 0.116949 | −1 | 0 | 1 | 0.82 | 63 |
| 72 | 0.117693 | −1 | 0 | 2 | 1.64 | 35 |
| 73 | 0.130158 | −1 | −0.2 | −7 | 1.06 | 17 |
| 74 | 0.131741 | −1 | 0.0999999 | −16 | 0.63 | 23 |
| 75 | 0.135592 | −1 | −0.5 | −11 | 1.89 | 27 |
| 76 | 0.00858709 | −1 | −0.3 | −2 | −0.0799999 | −33 |
| 77 | 0.133451 | −1 | 0.4 | −26 | 0.9 | 65 |
| 78 | 0.0889569 | −1 | −0.7 | 5 | 0.81 | −28 |
| 79 | 0.117563 | −1 | 0.2 | 8 | 0.74 | 10 |
| 80 | 0.0476948 | −1 | 0.1 | 1 | 2.16 | −11 |
| 81 | 0.103679 | −1 | −0.4 | −2 | −0.76 | 51 |
| 82 | 0.0782636 | −1 | −0.3 | −5 | 1.28 | 13 |
| 83 | 0.28664 | −1 | −0.6 | 1 | 0.54 | 17 |
| 84 | 0.132771 | −1 | −0.3 | −14 | −0.43 | −29 |
| 86 | 0.126602 | −1 | 0 | 0 | −3.42 | 10 |
| 87 | 0.122502 | −1 | −0.0999999 | −4 | 4.44 | 15 |
| 88 | 0.134354 | −1 | −0.2 | −1 | 1.43 | −37 |
| 90 | 0.0306998 | −1 | −0.0999999 | 0 | 2.14 | −21 |
| 91 | 0.0941547 | −1 | 0.3 | −3 | −0.42 | 49 |
| 92 | 0.152033 | −1 | −0.5 | 2 | 0.42 | −28 |
| 93 | 1.03441 | −1 | −0.0999999 | 1 | −0.35 | 18 |
| 94 | 0.0659902 | −1 | 0.6 | −3 | 1.1 | 5 |
| 96 | 0.132097 | −1 | −0.6 | −17 | −0.64 | −64 |
| 97 | 0.0657166 | −1 | −0.4 | 2 | 0.61 | 11 |
| 99 | 0.105808 | −1 | 0 | 22 | 0.52 | −6 |
| 100 | 0.055372 | −1 | 0.0999999 | 4 | 0.48 | −7 |
| 101 | 0.0745408 | −1 | 0.1 | 3 | 3.06 | −10 |
| 102 | 0.0707876 | −1 | −0.3 | 4 | −1.38 | −28 |
| 103 | 0.103869 | −1 | 0.0999999 | 4 | −0.150001 | −25 |
| 104 | 0.0616809 | −1 | 0.3 | −3 | 2.99 | −9 |
| 105 | 0.0305108 | −1 | 0 | 1 | 0.78 | −13 |
| 106 | 0.022998 | −1 | 0 | 3 | −0.5 | 2 |
| 107 | 0.105197 | −1 | 0.5 | 1 | −3.77 | −8 |
| 108 | 0.111541 | −1 | 0.6 | −2 | 3.21 | 9 |
| 109 | 0.0417176 | −1 | 0.7 | 1 | 0.0599999 | −11 |
| 110 | 0.136878 | −1 | 0.0999999 | −14 | 1.95 | −5 |
| 111 | 0.128737 | −1 | 0.5 | −9 | −2.76 | 36 |
| 112 | 0.126936 | −1 | 0.2 | −2 | −4.04 | −40 |
| 113 | 0.130452 | −1 | −0.3 | −4 | −0.429999 | 42 |
| 114 | 0.134852 | −1 | 1 | 3 | 0.5 | −39 |
| 115 | 0.133611 | −1 | 0 | −4 | −2.45 | −15 |
| 116 | 0.134519 | −1 | −0.2 | 6 | −3.22 | −9 |
| 117 | 0.130499 | −1 | 0.2 | 1 | −2.57 | −42 |
| 118 | 0.137301 | −1 | −0.3 | 0 | −0.0600004 | −62 |
| 119 | 0.137353 | −1 | −0.3 | −23 | 4.33 | 6 |
| 120 | 0.0861764 | −1 | −0.2 | −4 | −1.02 | 7 |
| 122 | 0.0700848 | −1 | −0.2 | 4 | 1.21 | −10 |
| 123 | 0.0751323 | −1 | −0.0999999 | 0 | −0.77 | −13 |
| 124 | 0.135359 | −1 | 0.2 | 8 | −0.85 | 39 |
| 125 | 0.0166803 | −1 | 0.5 | 2 | 0.179999 | −10 |
| 126 | 0.131763 | −1 | −0.4 | 7 | −2.91 | −72 |
| 127 | 0.0172782 | −1 | 0.1 | −1 | −0.25 | −13 |
| 128 | 0.11654 | −1 | 0.1 | 3 | −3.1 | −2 |
| 129 | 0.0927854 | −1 | 0.0999999 | −7 | −0.59 | 10 |
| 130 | 0.645186 | −1 | −0.2 | 1 | −2.03 | 16 |
| 131 | 0.136789 | −1 | 0.2 | −13 | −4.52 | 7 |
| 132 | 0.873042 | −1 | 0.4 | −5 | −0.11 | −1 |
| 133 | 0.0756838 | −1 | −0.7 | 0 | 0.89 | −8 |
| 134 | 0.0602228 | −1 | 0.3 | 5 | 1.42 | −6 |
| 135 | 0.0412774 | −1 | 0 | 1 | 0.9 | −17 |
| 137 | 0.130827 | −1 | 0.4 | 7 | 2.1 | 14 |
| 138 | 1.1236 | −1 | 0.5 | 3 | 3.5 | −3 |
| 139 | 0.1278 | −1 | −0.8 | 2 | −4.7 | −18 |
| 140 | 0.10774 | −1 | −0.3 | 2 | 3.1 | −16 |
| 144 | 0.114813 | −1 | 0 | −2 | −3.2 | −18 |
| 145 | 0.133279 | −1 | −0.4 | −30 | 8.8 | 10 |
| 146 | 0.120162 | −1 | 0.4 | −8 | 1.2 | −13 |
| 147 | 0.104482 | −1 | −0.2 | 24 | 1.8 | −5 |
| 148 | 0.0371389 | −1 | 1 | 0 | 1.2 | 9 |
| 149 | 0.136766 | −1 | −0.4 | −4 | 1.8 | −55 |
| 150 | 0.129676 | −1 | 0.3 | −11 | −0.8 | −14 |
| 151 | 0.145706 | −1 | −0.2 | −2 | 0.4 | −31 |
| 152 | 0.0353121 | −1 | 0.2 | −3 | −1 | −23 |
| 153 | 0.109666 | −1 | 0 | −1 | 3.3 | −20 |
| 155 | 0.0790375 | −1 | −0.2 | 1 | 1 | 7 |
| 156 | 0.134073 | −1 | 0.5 | 5 | 8.5 | 100 |
| 157 | 0.136042 | −1 | 0.2 | 5 | 1.4 | 66 |
| 158 | 0.106444 | −1 | 1.1 | −1 | 2.6 | −16 |
| 159 | 0.135323 | −1 | −0.2 | −3 | 4.5 | 27 |
| 160 | 0.102584 | −1 | 0.4 | −6 | 1.9 | 5 |
| 161 | 0.133333 | −1 | 0.3 | −4 | 6.1 | 46 |
| 162 | 0.414134 | −1 | 0 | 2 | 1.9 | −2 |
| 163 | 0.431208 | −1 | 0 | −6 | 1.5 | −6 |
| 164 | 0.115314 | −1 | 0.2 | 5 | 2.2 | 10 |
| 165 | 0.0358967 | −1 | 1.1 | 3 | 2.2 | 3 |
| 166 | 0.118849 | −1 | −0.5 | −6 | 1.7 | 34 |
| 167 | 0.847278 | −1 | −0.6 | 2 | 3.3 | 1 |
| 168 | 0.133811 | −1 | −0.0999999 | −44 | −0.3 | 6 |
| 169 | 0.132909 | −1 | −0.2 | 11 | 3.3 | 28 |
| 170 | 0.061998 | −1 | −0.2 | −2 | 3.1 | −8 |
| 171 | 0.137185 | −1 | 0.0999999 | 1 | 5.9 | 59 |
| 172 | 0.125603 | −1 | 0.2 | 0 | 5.3 | −13 |
| 175 | 2.06678 | 1 | 0.9 | 0 | −0.0900002 | 16 |
| 176 | 1.86131 | 1 | −0.0999999 | −7 | 0.23 | −18 |
| 177 | 2.04559 | 1 | 0.8 | 2 | −0.02 | −32 |
| 178 | 1.861 | 1 | 0 | −29 | 0.650001 | −12 |
| 179 | 2.94952 | 1 | 0.7 | 13 | −0.63 | 5 |
| 180 | 1.85829 | 1 | 0 | −10 | 3.91 | −20 |
| 181 | 1.86414 | 1 | 0.3 | −27 | 1.9 | −27 |
| 182 | 1.2058 | 1 | 0.4 | 5 | 3.7 | −1 |
| 183 | 2.2239 | 1 | 0 | 3 | 3.8 | −1 |
| 184 | 1.86419 | 1 | −0.3 | −31 | 1.2 | 4 |
| 185 | 2.14876 | 1 | 0.4 | −1 | −0.8 | 18 |
| 186 | 2.21883 | 1 | 0.0999999 | −6 | 0.5 | −3 |

The separating surface is represented by:

$${\sum }_{n = 1}^{k} {α}_{n} K ( {x}_{,} {s}_{n} ) {y}_{n} + b = 0 ,$$

where,
k=165 is the number of support vectors,
αₙ is the Lagrange parameter or multiplier for the n^{th} patient (given in the second column)
yₙ is the class label for the n^{th} patient (given in the third column),
b is the offset (SVM parameter), and
K(x,sₙ) is the kernel function for the n^{th} patient defined as:
   K(x,sₙ)=e^{M}
in which:

$$M = {\sum }_{l = 1}^{4} {( {x}_{i} - d ( n , i ) )}^{2} / σ$$

Where,
d(n,i),i=1,2, . . . ,4 are the values in columns 4 through 7,
xᵢ is a new vector to be classified, such as from the validation set; and

σ is sigma as a user settable parameter.

A value for σ used in one embodiment is as defined in the SVM parameters above.

It should be noted that in the foregoing and other examples, the number of support vectors, the particular vectors in the training data set that are the support vectors, the Lagrange multipliers, and the offset are determined as a result of training. The Gaussian kernel function is a particular type of defined and known kernel function as described in Nello Cristianini and John Shawe-Taylor: An introduction to Support Vector Machines, Cambridge University Press, 2000. This SVM embodiment, and others described herein, use the known kernel function with the difference parameters as described herein.

Following are results obtained using the foregoing first example SVM represented in the following confusion matrix.

The following are results obtained using the above trained and validated SVM. The confusion matrix represents a summary of the predictive results recorded at step 218, for example, as a result of the testing step 214 of flowchart. It should be noted that the confusion matrix in this and other example SVM embodiments represent the results of executing flowchart 200 for 1000 cycles which results in testing class 2 patients 12,000 times. Recall that each of the 12 class 2 patients are tested once in each cycle of 6 iterations of the steps of flowchart 200.

| | | PREDICTED CLASS | | |
|---|---|---|---|---|
| | | class 1 | class 2 | Accuracy |
| TRUE | class 1 | 174165 | 837 | 99.52% |
| CLASS | class 2 | 11202 | 798 | 6.65% |

The foregoing confusion matrix states that there are a total of 174165+837=175002 instances of actual class 1 patients of which 837 were falsely, classified as being class 1. There are a total of 12202+798=12000 actual class 2 patients of which 11202 were falsely classified as being in class 2.

In a second example of an embodiment of an SVM, the following ten difference parameters: potassium, SGOT, SGPT, glycosylated haemoglobin, cholesterol, chloride, LDL, total proteins, phosphate and calcium were selected. Selection of the foregoing parameters were determined using ANOVA, matrix plots and intuition based on experience and empirical results.

The following internal SVM parameters were produced as a result of the SVM training and validation executing the processing steps of flowchart 200 of FIG. 8 using the foregoing 10 difference parameters for the collected input data for the 187 patients:

| Kernel Type | gaussian |
|---|---|
| Sigma | 6140.0 |
| Offset | −2.23207 |
| Number of support vectors | 42 |

The following second table includes the difference parameters for the support vectors determined. Each row in the table corresponds to data for one support vector. Columns 1-3 include data organized as described in connection with the first table of the first SVM embodiment example. The remaining columns correspond to the values for the 10 difference parameters.

| PT- | | | | | | | | | | | | |
|---|---|---|---|---|---|---|---|---|---|---|---|---|
| ID | lagranges | CL | K | Alt | Ast | HBA1C | Chol | Cl | LDL | TP | PO₄ | Ca |
| 7 | 24.8512 | −1 | 0.0999999 | 14 | 8 | −0.0599995 | 4 | −0.599998 | 8.8 | 0.4 | −0.4 | −0.2 |
| 8 | 100 | −1 | 0.3 | −9 | −5 | −1.15 | −33 | 2.6 | −27.2 | −0.4 | 0.0999999 | −0.8 |
| 11 | 23.4825 | −1 | 0.3 | −55 | −20 | 0.62 | 12 | −0.900002 | 14.8 | 0.8 | −0.4 | −0.3 |
| 16 | 34.6397 | −1 | 0.4 | 5 | 3 | −0.66 | 2 | 1.4 | 3.6 | 0.8 | 0.0999999 | 1.3 |
| 29 | 25.7872 | −1 | 0 | 15 | 9 | 1.68 | 14 | −5.2 | 31.4 | 1 | −0.0999999 | 0.700001 |
| 30 | 14.7238 | −1 | 0.2 | 0 | 2 | −1.98 | −1 | 4 | −0.599998 | 0.7 | 1 | 1.1 |
| 32 | 7.34327 | −1 | −0.4 | −6 | −2 | −1.47 | 5 | 2.8 | 9.2 | −0.1 | 1.3 | 0.4 |
| 40 | 36.0188 | −1 | 0.2 | −7 | −6 | 2.11 | −36 | −2 | −29.8 | 0.0999994 | 0.2 | −1.2 |
| 42 | 100 | −1 | −0.3 | −19 | −9 | −1.78 | −4 | −2.3 | −22 | 0.0999994 | −0.6 | −1.7 |
| 51 | 100 | −1 | 0.2 | −14 | −3 | −0.2 | 2 | 3.9 | 3.8 | −0.3 | −0.8 | −0.799999 |
| 60 | 66.6024 | −1 | 0 | 13 | 5 | 0.61 | 24 | 1.3 | 9.8 | 0.7 | 0.4 | −0.4 |
| 62 | 28.3199 | −1 | 0.6 | 1 | −2 | 2.52 | 12 | 1.6 | 9.2 | 0.2 | 0.2 | −0.299999 |
| 84 | 19.178 | −1 | −0.3 | −14 | −3 | −0.43 | −29 | −0.400002 | 2.8 | −0.0999994 | −0.5 | −0.1 |
| 86 | 18.6703 | −1 | 0 | 0 | 1 | −3.42 | 10 | 2.9 | 10.8 | 0.8 | −0.4 | 1.1 |
| 90 | 19.024 | −1 | −0.0999999 | 0 | −3 | 2.14 | −21 | 2.1 | −24.2 | 0.2 | −0.3 | 0.3 |
| 110 | 100 | −1 | 0.0999999 | −14 | −9 | 1.95 | −5 | 1.9 | 0.800003 | 0.2 | −0.5 | −2.4 |
| 119 | 5.68557 | −1 | −0.3 | −23 | −1 | 4.33 | 6 | 0.5 | −38.4 | 0.7 | −0.2 | 0.6 |
| 130 | 13.6486 | −1 | −0.2 | 1 | 0 | −2.03 | 16 | −0.800003 | 16.6 | −0.2 | −0.1 | −0.200001 |
| 138 | 35.6026 | −1 | 0.5 | 3 | 8 | 3.5 | −3 | 1 | 11.6 | −0.400001 | −0.1 | −0.1 |
| 143 | 8.71654 | −1 | 0.3 | 0 | 7 | 4 | 3 | −2 | 12.6 | 0.0999999 | 1 | 0.1 |
| 145 | 39.6365 | −1 | −0.4 | −30 | −9 | 8.8 | 10 | −6 | 10.4 | 0 | −0.2 | 0.299999 |
| 146 | 98.2635 | −1 | 0.4 | −8 | −6 | 1.2 | −13 | −1 | −4.40001 | 0.0999999 | 0.3 | −0.0999994 |
| 147 | 24.3469 | −1 | −0.2 | 24 | 15 | 1.8 | −5 | −1 | −0.400009 | −0.9 | −0.5 | −0.1 |
| 153 | 53.5136 | −1 | 0 | −1 | 4 | 3.3 | −20 | −4 | −40.4 | −0.2 | 0.3 | 0 |
| 158 | 8.77936 | −1 | 1.1 | −1 | 6 | 2.6 | −16 | 4 | −5.6 | 0.299999 | −0.3 | −0.8 |
| 160 | 35.2869 | −1 | 0.4 | −6 | 0 | 1.9 | 5 | 2 | −6 | −0.3 | 0.4 | 0 |
| 164 | 10.636 | −1 | 0.2 | 5 | 2 | 2.2 | 10 | −2 | 11.8 | −0.1 | 0.0999999 | −0.4 |
| 165 | 65.4638 | −1 | 1.1 | 3 | −1 | 2.2 | 3 | 1 | 5.2 | 0.6 | 0.7 | −0.2 |
| 172 | 21.1241 | −1 | 0.2 | 0 | 0 | 5.3 | −13 | −3 | −26.6 | 0.1 | 0.6 | 0.3 |
| 174 | 1.88169 | −1 | 0.0999999 | 0 | 3 | 2.1 | −5 | 1 | −4.8 | −0.6 | 0.2 | 0.2 |
| 175 | 100 | 1 | 0.9 | 0 | 0 | −0.0900002 | 16 | −0.5 | 2 | 1 | 0.4 | 1.1 |
| 176 | 100 | 1 | −0.0999999 | −7 | −1 | 0.23 | −18 | 1.4 | 2.4 | −0.3 | −0.7 | −0.2 |
| 177 | 100 | 1 | 0.8 | 2 | 0 | −0.02 | −32 | 1.1 | −39.8 | 0.2 | 0.2 | −0.200001 |
| 178 | 100 | 1 | 0 | −29 | −8 | 0.650001 | −12 | −2.8 | −9.4 | 0.2 | 0.1 | −0.599999 |
| 179 | 100 | 1 | 0.7 | 13 | 7 | −0.63 | 5 | 1.8 | 11.8 | −0.2 | −0.2 | −1.5 |
| 180 | 100 | 1 | 0 | −10 | −5 | 3.91 | −20 | 1.3 | −28 | 0 | 1.4 | −0.5 |
| 181 | 41.2267 | 1 | 0.3 | −27 | −15 | 1.9 | −27 | −2 | −18.8 | 0 | 0.3 | −0.7 |
| 182 | 100 | 1 | 0.4 | 5 | 4 | 3.7 | −1 | 2 | 2 | −0.1 | −0.1 | −0.900001 |
| 183 | 100 | 1 | 0 | 3 | −1 | 3.8 | −1 | 2 | 3.8 | 0.0999994 | 0.1 | −1.3 |
| 184 | 100 | 1 | −0.3 | −31 | −15 | 1.2 | 4 | 3 | 3.59999 | −0.9 | −0.2 | −0.0999994 |
| 185 | 100 | 1 | 0.4 | −1 | 0 | −0.8 | 18 | −3 | 19.8 | 0.3 | −0.3 | 0.8 |
| 186 | 100 | 1 | 0.0999999 | −6 | −2 | 0.5 | −3 | −1 | −16.8 | −0.0999994 | 0.1 | 0.5 |

The separating surface corresponding to the above may be represented by:

$${\sum }_{n = 1}^{k} {α}_{n} K ( x , {s}_{n} ) {y}_{n} + b = 0 ,$$

where,
k=42 is the number of support vectors,
αₙ is the Lagrange parameter for the n^{th} patient,
yₙ is the class label for the n^{th} patient,
b is the offset,
   and
K(x,sₙ) is the kernel function for the n^{th} patient defined as:
   K(x,sₙ)=e^{M}
where,

$$M = {\sum }_{i = 1}^{10} {( {x}_{i} - d ( n , i ) )}^{2} / σ$$

and
d(n,i),i=1,2, . . . ,10 are the values in columns 4 through 13 of the previous table corresponding to the difference parameter values.

Following are results obtained using the above second embodiment of the trained and validated SVM as recorded, for example, at during various iterations of step 218:

| | | PREDICTED CLASS | | |
|---|---|---|---|---|
| | | class 1 | class 2 | Accuracy |
| TRUE | class 1 | 173587 | 1413 | 99.19% |
| CLASS | class 2 | 10605 | 1395 | 11.62% |

Overall accuracy 93.57%

The foregoing confusion matrix states that there are a total of 173587+1413=175000 instances of actual class 1 patients of which 1413 were falsely classified as belonging to class 2.

In a third example SVM embodiment, the following six difference parameters: cholesterol, chloride, LDL, total proteins, phosphate and calcium were selected. Selection of the foregoing parameters was determined using ANOVA, matrix plots and intuition.

The following internal SVM parameters were produced as a result of the SVM training and validation by executing the processing steps of flowchart 200 of FIG. 8 using the foregoing 10 difference parameters for the collected input data for the 187 patients:

| Kernel Type | gaussian |
|---|---|
| Sigma | 5.0 |
| Offset | −0.878728 |
| Number of support vectors | 179 |

The following third table includes difference parameters for each of the support vectors determined as a result of training. The third table is organized similarly to the first and second tables as described herein. In particular, columns 1-3 include data as described above for each support vector. The remaining columns of each row include difference parameter values for each of the support vectors corresponding to each row.

| PT-ID | Lagrange | Cl | Chol | Cl | LDL | TP | PO₄ | Ca |
|---|---|---|---|---|---|---|---|---|
| 0 | 0.126377 | −1 | −32 | −3.2 | −37 | −0.7 | −0.2 | −0.700001 |
| 1 | 0.099679 | −1 | −9 | −3.4 | −4.2 | 0.1 | −0.3 | −1.8 |
| 2 | 0.118697 | −1 | 25 | −3.6 | 28.8 | 0.5 | −0.6 | −1.1 |
| 3 | 0.123535 | −1 | −47 | −1.3 | −13.8 | 0.2 | 0.1 | −1.7 |
| 4 | 0.0488012 | −1 | −22 | −2.8 | −19.8 | 0.2 | −0.0999999 | −0.1 |
| 5 | 0.0807789 | −1 | −4 | −3.5 | 0.599998 | 0.3 | −0.7 | −0.700001 |
| 6 | 0.102998 | −1 | −24 | −0.5 | −9.2 | 0.5 | −0.2 | −0.1 |
| 7 | 0.1408 | −1 | 4 | −0.599998 | 8.8 | 0.4 | −0.4 | −0.2 |
| 8 | 0.118211 | −1 | −33 | 2.6 | −27.2 | −0.4 | 0.0999999 | −0.8 |
| 9 | 0.149318 | −1 | 19 | −0.900002 | 5 | 0.2 | 0.4 | 0.4 |
| 10 | 0.101129 | −1 | −5 | −3.3 | −2.8 | −0.200001 | 0.0999999 | 0 |
| 11 | 0.0561103 | −1 | 12 | −0.900002 | 14.8 | 0.8 | −0.4 | −0.3 |
| 12 | 0.051422 | −1 | −1 | −0.199997 | −1.8 | −0.5 | −0.5 | −0.700001 |
| 13 | 0.0754909 | −1 | −21 | −1.1 | −8.60001 | −0.5 | −0.7 | −0.599999 |
| 14 | 0.124324 | −1 | 39 | −2.2 | −13.8 | 1 | −0.3 | 1.5 |
| 15 | 0.11689 | −1 | 23 | 0.800003 | 6.39999 | 0.5 | 0 | 1.4 |
| 16 | 0.18501 | −1 | 2 | 1.4 | 3.6 | 0.8 | 0.0999999 | 1.3 |
| 18 | 0.121873 | −1 | 93 | −1.1 | 46.6 | 1.2 | −0.0999999 | 0.5 |
| 19 | 0.111891 | −1 | 4 | −2.5 | 15.8 | 1.6 | 0.4 | 0.3 |
| 20 | 0.122082 | −1 | 36 | 1.6 | 66.4 | 1.3 | −0.5 | 0.5 |
| 21 | 0.122402 | −1 | 3 | −1.4 | 49 | 0.2 | 0.3 | 1.2 |
| 22 | 0.105506 | −1 | 6 | 0.400002 | 24.4 | 0.9 | 0 | 0.2 |
| 23 | 0.125248 | −1 | 37 | −0.400002 | 27.2 | −0.0999999 | 0.2 | 0 |
| 24 | 1.22518 | −1 | 17 | −2 | 18.6 | 0.3 | −0.0999999 | 0.0999994 |
| 25 | 0.117169 | −1 | 26 | −0.0999985 | 15.6 | 0 | 0.7 | 0.400001 |
| 26 | 0.0540365 | −1 | 13 | −1.8 | 13.8 | 0.4 | 0.2 | −0.0999994 |
| 27 | 0.11858 | −1 | −6 | 0.300003 | 66.2 | 0.5 | 0.3 | 0 |
| 28 | 0.124583 | −1 | 120 | 1 | 99.2 | −0.3 | 0.2 | −0.6 |
| 29 | 0.121283 | −1 | 14 | −5.2 | 31.4 | 1 | −0.0999999 | 0.700001 |
| 30 | 0.145444 | −1 | −1 | 4 | −0.599998 | 0.7 | 1 | 1.1 |
| 31 | 0.120823 | −1 | 62 | 2 | −105.4 | 0.7 | 0.8 | 0.2 |
| 32 | 0.242645 | −1 | 5 | 2.8 | 9.2 | −0.1 | 1.3 | 0.4 |
| 33 | 0.0887115 | −1 | 9 | −1 | 7.2 | 0.2 | −0.0999999 | 0.599999 |
| 34 | 0.0793491 | −1 | 6 | −3.7 | −1.2 | 0.299999 | −0.0999999 | 0.400001 |
| 35 | 0.0587891 | −1 | 13 | 0.400002 | 10.8 | −0.400001 | −0.0999999 | −0.6 |
| 36 | 0.104305 | −1 | −1 | −0.300003 | −4.60001 | 0.2 | 1 | 0.0999994 |
| 37 | 0.0784003 | −1 | −7 | −0.199997 | 2.39999 | 0.4 | −0.2 | −1 |
| 38 | 0.103174 | −1 | 1 | −0.0999985 | 9 | 0.2 | 0.3 | −1.1 |
| 39 | 0.110331 | −1 | −18 | −0.699997 | −18.4 | 0.2 | 0.6 | −1.3 |
| 40 | 0.123825 | −1 | −36 | −2 | −29.8 | 0.0999994 | 0.2 | −1.2 |
| 41 | 0.12255 | −1 | 24 | −0.699997 | 19.2 | 0.7 | 0.3 | −1.1 |
| 42 | 0.102185 | −1 | −4 | −2.3 | −22 | 0.0999994 | −0.6 | −1.7 |
| 43 | 0.120759 | −1 | 46 | 2 | 40.2 | −0.1 | −0.0999999 | −1.5 |
| 44 | 0.124176 | −1 | −33 | 3.2 | −33 | −0.0999999 | −0.2 | −1.2 |
| 45 | 0.122721 | −1 | −50 | 7 | −40.4 | −0.1 | −0.9 | −1.6 |
| 46 | 0.116019 | −1 | 31 | −0.699997 | 22.8 | 0.5 | 0.8 | −1.4 |
| 47 | 0.0760766 | −1 | 14 | −0.400002 | 9.4 | 0.7 | 0.4 | −1.2 |
| 48 | 0.068014 | −1 | 11 | −0.5 | 5.4 | −0.1 | 0 | −1.9 |
| 49 | 0.106345 | −1 | 3 | −3.6 | −9.8 | 0.2 | 0.5 | −0.9 |
| 50 | 0.120026 | −1 | 128 | −1.3 | 19.6 | 0.400001 | −0.0999999 | −1.2 |
| 51 | 0.914103 | −1 | 2 | 3.9 | 3.8 | −0.3 | −0.8 | −0.799999 |
| 52 | 0.0751068 | −1 | 18 | −1.7 | 12 | 0.2 | 0 | −0.7 |
| 53 | 0.118424 | −1 | 44 | 0.199997 | 20.8 | 0.8 | −0.2 | −1 |
| 54 | 0.101712 | −1 | −23 | 0.599998 | −20 | 0.200001 | 0.3 | −0.5 |
| 55 | 0.0865527 | −1 | 3 | 0.0999985 | 0 | 0.8 | 0.5 | −0.299999 |
| 56 | 0.0127582 | −1 | −13 | 1.6 | −10.8 | 0.400001 | −1 | −0.4 |
| 57 | 0.122114 | −1 | −12 | −1.8 | −20 | 0.3 | −0.4 | −1.8 |
| 58 | 0.118346 | −1 | 26 | −1.9 | 4.4 | 0.2 | −0.4 | −0.0999994 |
| 59 | 0.0988454 | −1 | 2 | −4.8 | −5.6 | 0.2 | 0.2 | −0.5 |
| 60 | 0.1163 | −1 | 24 | 1.3 | 9.8 | 0.7 | 0.4 | −0.4 |
| 61 | 0.11729 | −1 | 53 | −4.9 | 25.6 | 0.9 | −0.0999999 | −0.2 |
| 62 | 0.0733102 | −1 | 12 | 1.6 | 9.2 | 0.2 | 0.2 | −0.299999 |
| 63 | 0.278505 | −1 | 18 | 0.599998 | 0 | 0.7 | −0.3 | −0.5 |
| 64 | 0.118815 | −1 | 61 | 0.300003 | 74.8 | 0.2 | 0.5 | −0.599999 |
| 65 | 0.477134 | −1 | −13 | −0.900002 | −11 | 0.2 | 0.9 | −1.1 |
| 67 | 0.0917121 | −1 | −3 | −3 | −7.8 | 0 | 0 | −1.4 |
| 68 | 0.921019 | −1 | −9 | −0.900002 | −25 | −0.200001 | −0.6 | −1.7 |
| 69 | 0.0579035 | −1 | −2 | −1.7 | −6.6 | −0.4 | −0.0999999 | −1.5 |
| 70 | 0.123706 | −1 | 47 | −5.2 | 25.2 | 0.8 | −0.5 | −0.9 |
| 71 | 0.124831 | −1 | 63 | −4.1 | 52.8 | −0.3 | −0.2 | −1.6 |
| 72 | 0.125199 | −1 | 35 | 0.699997 | 78.4 | 0.0999999 | −1.1 | −1.4 |
| 73 | 0.129224 | −1 | 17 | 0.400002 | 4.6 | −0.7 | −0.5 | −2.1 |
| 74 | 0.125509 | −1 | 23 | −0.900002 | −50.8 | −0.8 | −0.3 | −2.6 |
| 75 | 0.123126 | −1 | 27 | −2.5 | 11.6 | −0.1 | −0.0999999 | −1.6 |
| 76 | 0.118175 | −1 | −33 | −0.300003 | −24.2 | −0.2 | 0.1 | −2.1 |
| 77 | 0.0791367 | −1 | 65 | −1 | 42.8 | 0.4 | 0.2 | −1.2 |
| 78 | 0.378417 | −1 | −28 | 0.599998 | −19.6 | −0.0999999 | 0.0999999 | −1.7 |
| 79 | 0.0504577 | −1 | 10 | 0.300003 | 2.79999 | −0.1 | 0.5 | −1.5 |
| 80 | 0.118559 | −1 | −11 | 1.6 | 1.6 | 0.3 | 0.2 | −1.5 |
| 81 | 0.118121 | −1 | 51 | −1.3 | 54.2 | 0.400001 | 0.1 | 0.799999 |
| 82 | 0.25834 | −1 | 13 | 0.199997 | 2.8 | 0.2 | −0.2 | −0.0999994 |
| 83 | 0.106044 | −1 | 17 | 0.800003 | 13 | 0.4 | −0.6 | 0.2 |
| 84 | 0.121452 | −1 | −29 | −0.400002 | 2.8 | −0.0999994 | −0.5 | −0.1 |
| 86 | 0.108607 | −1 | 10 | 2.9 | 10.8 | 0.8 | −0.4 | 1.1 |
| 87 | 0.119705 | −1 | 15 | 0.699997 | −7.6 | −0.7 | −0.3 | −0.3 |
| 88 | 0.118286 | −1 | −37 | −0.599998 | −22.8 | 0 | −0.1 | −0.4 |
| 89 | 0.12426 | −1 | 18 | −2.2 | 35.8 | 0.0999999 | −0.8 | −0.5 |
| 90 | 0.157725 | −1 | −21 | 2.1 | −24.2 | 0.2 | −0.3 | 0.3 |
| 91 | 0.119687 | −1 | 49 | −0.5 | 46.8 | 0.3 | −0.9 | 0.5 |
| 92 | 0.136232 | −1 | −28 | 0.300003 | −21.4 | 0.4 | −0.3 | 0.4 |
| 93 | 0.0878501 | −1 | 18 | −1.2 | 9.8 | 0.1 | −0.5 | −0.3 |
| 94 | 0.193191 | −1 | 5 | −0.400002 | 2.8 | 0.3 | 0.1 | −0.1 |
| 95 | 0.0708323 | −1 | −21 | 0.699997 | −8.39999 | −0.3 | −0.4 | −0.2 |
| 96 | 0.120987 | −1 | −64 | 3.9 | 63.4 | −0.3 | −0.8 | −0.7 |
| 98 | 0.129091 | −1 | 13 | 0 | −1.8 | 0.3 | 0 | −0.200001 |
| 99 | 0.119225 | −1 | −6 | −4.5 | 7 | 0.6 | 0.6 | 0.2 |
| 100 | 0.0942736 | −1 | −7 | 0.199997 | −25.2 | 0.3 | −0.6 | −0.7 |
| 101 | 0.0831761 | −1 | −10 | −1.2 | −3.59999 | 0.0999994 | −0.4 | −0.7 |
| 102 | 0.113337 | −1 | −28 | −1 | −24.6 | 0.3 | 0.1 | −0.7 |
| 103 | 0.120274 | −1 | −25 | 2.2 | −12.2 | −0.0999999 | 0.2 | −1 |
| 104 | 0.119777 | −1 | −9 | −3.4 | −44.2 | 0.3 | −0.7 | −1 |
| 105 | 0.109525 | −1 | −13 | 1.7 | −7 | 0 | 0.0999999 | −1.4 |
| 106 | 0.10085 | −1 | 2 | −2.3 | 5.39999 | −0.2 | −0.0999999 | −1.5 |
| 107 | 0.118753 | −1 | −8 | 7.3 | −3.8 | −0.0999999 | −0.3 | −1.5 |
| 108 | 0.115008 | −1 | 9 | 0.0999985 | 24.4 | 0.2 | 0 | −2 |
| 109 | 0.114865 | −1 | −11 | 2.9 | −13.4 | 0.2 | 0 | −2 |
| 110 | 0.148307 | −1 | −5 | 1.9 | 0.800003 | 0.2 | −0.5 | −2.4 |
| 111 | 0.124932 | −1 | 36 | −0.5 | −6.6 | 0.3 | 0.4 | −0.9 |
| 112 | 0.115749 | −1 | −40 | −0.199997 | −25 | −0.2 | −1.2 | −2 |
| 113 | 0.123494 | −1 | 42 | −0.5 | 16.8 | −0.2 | 0.4 | −2 |
| 114 | 0.119721 | −1 | −39 | 4.6 | −31.2 | −0.0999999 | −0.3 | −2 |
| 115 | 0.121758 | −1 | −15 | 1.6 | −21.6 | −0.0999994 | 0 | 1.5 |
| 116 | 0.11768 | −1 | −9 | 4.9 | −3.4 | 0.0999999 | 0.2 | 1.7 |
| 117 | 0.120119 | −1 | −42 | −1.7 | −31.6 | 0 | −0.5 | 1.2 |
| 118 | 0.124026 | −1 | −62 | −0.599998 | −41.8 | −0.2 | 0 | −0.5 |
| 119 | 0.123562 | −1 | 6 | 0.5 | −38.4 | 0.7 | −0.2 | 0.6 |
| 120 | 0.0867245 | −1 | 7 | −2.9 | −2.40001 | −0.0999994 | −0.3 | −0.3 |
| 121 | 0.120943 | −1 | −11 | −0.300003 | −46.4 | 0 | −0.4 | 0.0999994 |
| 122 | 0.53758 | −1 | −10 | −1 | −9.8 | 0.1 | 0.3 | −0.3 |
| 123 | 0.115765 | −1 | −13 | 3.4 | −2 | −0.5 | 0.2 | −0.400001 |
| 124 | 0.12397 | −1 | 39 | −2.4 | 4 | 0.5 | 0 | −3.7 |
| 125 | 0.125318 | −1 | −10 | 3.9 | 12.4 | −0.4 | 0.4 | −3.6 |
| 126 | 0.125509 | −1 | −72 | 3.6 | −45.6 | −0.5 | 0.0999999 | −4.3 |
| 127 | 0.0590771 | −1 | −13 | −0.300003 | −3.2 | 0.4 | 0 | 0.400001 |
| 128 | 0.202361 | −1 | −2 | −0.699997 | −20.4 | 0.0999999 | −0.1 | −0.4 |
| 129 | 0.0913021 | −1 | 10 | −2.9 | 3.2 | 0.2 | 0 | −0.1 |
| 131 | 0.136385 | −1 | 7 | 3 | 15.8 | −0.0999999 | 0 | 0.0999994 |
| 132 | 3.01489 | −1 | −1 | 1.1 | 1.8 | 0 | 0.4 | −0.2 |
| 133 | 0.116225 | −1 | −8 | 3.7 | 5.2 | 0.5 | 0.2 | −0.3 |
| 134 | 0.0702535 | −1 | −6 | 1.4 | 3 | 0.0999999 | −0.3 | −0.3 |
| 135 | 0.120219 | −1 | −17 | 2.1 | −10 | 0.4 | −0.2 | −0.4 |
| 136 | 0.06821 | −1 | −4 | 0 | −6.6 | 0.3 | 0.2 | 0 |
| 137 | 0.113958 | −1 | 14 | −1 | 6.40001 | 0.7 | 0.2 | 0.6 |
| 138 | 0.120431 | −1 | −3 | 1 | 11.6 | −0.400001 | −0.1 | −0.1 |
| 139 | 0.0590948 | −1 | −18 | 0 | −14.2 | −0.2 | 0.3 | −0.8 |
| 140 | 0.119397 | −1 | −16 | −4 | −18.8 | −0.2 | 0.2 | −0.2 |
| 142 | 0.0598162 | −1 | 2 | −4 | −7.59999 | −0.2 | −0.3 | −0.0999994 |
| 143 | 0.129876 | −1 | 3 | −2 | 12.6 | 0.0999999 | 1 | 0.1 |
| 144 | 0.0782049 | −1 | −18 | −1 | −14.4 | −0.3 | 0.2 | 0 |
| 145 | 0.116778 | −1 | 10 | −6 | 10.4 | 0 | −0.2 | 0.299999 |
| 146 | 0.0751736 | −1 | −13 | −1 | −4.40001 | 0.0999999 | 0.3 | −0.0999994 |
| 147 | 0.0792525 | −1 | −5 | −1 | −0.400009 | −0.9 | −0.5 | −0.1 |
| 148 | 0.0568049 | −1 | 9 | −2 | 5.4 | 0.6 | 0.8 | −0.1 |
| 149 | 0.117594 | −1 | −55 | −1 | −10.4 | −0.2 | 0.6 | −0.599999 |
| 150 | 0.121184 | −1 | −14 | −6 | 12.6 | 0.299999 | −0.2 | −0.5 |
| 151 | 0.154819 | −1 | −31 | −1 | −18.6 | −0.2 | −0.6 | −0.6 |
| 152 | 0.11094 | −1 | −23 | −2 | −20.4 | −0.0999999 | −0.0999999 | 0.0999994 |
| 153 | 0.125046 | −1 | −20 | −4 | −40.4 | −0.2 | 0.3 | 0 |
| 154 | 0.0918141 | −1 | 10 | 2 | 4.2 | −0.3 | −1.1 | −1 |
| 156 | 0.123946 | −1 | 100 | 1 | 95 | 1 | 0.1 | 0.5 |
| 157 | 0.0661654 | −1 | 66 | −1 | 42.4 | 0.400001 | −0.1 | −0.3 |
| 158 | 0.116929 | −1 | −16 | 4 | −5.6 | 0.299999 | −0.3 | −0.8 |
| 159 | 0.117427 | −1 | 27 | 0 | 24 | 0.7 | 1.2 | −0.8 |
| 160 | 0.12265 | −1 | 5 | 2 | −6 | −0.3 | 0.4 | 0 |
| 161 | 0.121974 | −1 | 46 | −1 | 42.4 | 0.8 | 1.1 | 0.900001 |
| 162 | 0.087889 | −1 | −2 | −4 | 1.6 | −0.9 | 0.2 | −1 |
| 164 | 0.106291 | −1 | 10 | −2 | 11.8 | −0.1 | 0.0999999 | −0.4 |
| 165 | 0.269721 | −1 | 3 | 1 | 5.2 | 0.6 | 0.7 | −0.2 |
| 166 | 0.117528 | −1 | 34 | −3 | 24.4 | −0.9 | −0.7 | −0.8 |
| 167 | 0.0775654 | −1 | 1 | −3 | 0.400009 | 0.1 | 0.0999999 | −0.5 |
| 168 | 0.113637 | −1 | 6 | −3 | 24 | −0.3 | 0.0999999 | −0.5 |
| 169 | 0.119706 | −1 | 28 | −3 | −1 | 0.2 | 0 | −0.3 |
| 170 | 0.119351 | −1 | −8 | −5 | −27.6 | −0.5 | 0.8 | −0.2 |
| 171 | 0.124829 | −1 | 59 | −1 | 53 | 0.5 | 0.4 | 0.7 |
| 172 | 0.123379 | −1 | −13 | −3 | −26.6 | 0.1 | 0.6 | 0.3 |
| 173 | 0.07415 | −1 | 13 | −2 | 15.8 | −0.2 | −0.9 | −0.2 |
| 174 | 0.0942835 | −1 | −5 | 1 | −4.8 | −0.6 | 0.2 | 0.2 |
| 175 | 1.92811 | 1 | 16 | −0.5 | 2 | 1 | 0.4 | 1.1 |
| 176 | 1.88094 | 1 | −18 | 1.4 | 2.4 | −0.3 | −0.7 | −0.2 |
| 177 | 1.88118 | 1 | −32 | 1.1 | −39.8 | 0.2 | 0.2 | −0.200001 |
| 178 | 2.09378 | 1 | −12 | −2.8 | −9.4 | 0.2 | 0.1 | −0.599999 |
| 179 | 1.89688 | 1 | 5 | 1.8 | 11.8 | −0.2 | −0.2 | −1.5 |
| 180 | 1.88311 | 1 | −20 | 1.3 | −28 | 0 | 1.4 | −0.5 |
| 181 | 1.9477 | 1 | −27 | −2 | −18.8 | 0 | 0.3 | −0.7 |
| 182 | 3.59991 | 1 | −1 | 2 | 2 | −0.1 | −0.1 | −0.900001 |
| 183 | 1.03001 | 1 | −1 | 2 | 3.8 | 0.0999994 | 0.1 | −1.3 |
| 184 | 2.20357 | 1 | 4 | 3 | 3.59999 | −0.9 | −0.2 | −0.0999994 |
| 185 | 2.43418 | 1 | 18 | −3 | 19.8 | 0.3 | −0.3 | 0.8 |
| 186 | 1.8932 | 1 | −3 | −1 | −16.8 | −0.0999994 | 0.1 | 0.5 |

The separating surface corresponding to the foregoing may be represented by:

$${\sum }_{n = 1}^{k} {α}_{n} K ( x , {s}_{n} ) {y}_{n} + b = 0 ,$$

in which:
k=179 is the number of support vectors,
αₙ is the Lagrange parameter for the n^{th} patient,
yₙ is the class label for the n^{th} patient,
b is the offset, and
K(x,sₙ) is the kernel function for the n^{th} patient defined as:
K(x,sₙ)=e^{M}
where,

$$M = {\sum }_{1}^{6} {( {x}_{i} - d ( n , i ) )}^{2} / σ$$

and
d(n,i),i=1,2, . . . ,6 are the values in columns 4 through 9.

The following are results obtained using the above trained and validated SVM as recorded in iterations of step 218:

| | | PREDICTED CLASS | | |
|---|---|---|---|---|
| | | class 1 | class 2 | Accuracy |
| TRUE | class 1 | 174172 | 828 | 99.53% |
| CLASS | class 2 | 10925 | 1075 | 8.96% |

The foregoing confusion matrix states that there are a total of 174172+828 instances of actual class 1 patients of which 828 were falsely classified as being in class 1.

In a fourth example SVM embodiment, the following six difference parameters: potassium, SGPT, glycosylated haemoglobin, cholesterol, chloride and LDL, were selected with the following SVM parameters:

| Kernel Type | gaussian |
|---|---|
| Maximum number of Iterations | 168300 |
| Sigma | 22.0 |
| Offset | −0.857502 |
| Number of support vectors | 162 |

The foregoing parameters were determined using ANOVA, matrix plots, and intuition.

The following fourth table includes data for support vectors determined in the fourth embodiment. The table is organized similar to the other three tables of support vector data described herein in which there is one support vector associated with each row of the table. Columns 1-3 of each row include data for each support vector as described in connection with other tables. The remaining columns includes difference parameter data for each support vector.

| | | | | Alt | | | | |
|---|---|---|---|---|---|---|---|---|
| PT-ID | Lagranges | CL | K | (SGPT) | HBA1C | Chol | Cl | LDL |
| 0 | 0.566083 | −1 | −0.0999999 | 2 | −2.51 | −32 | −3.2 | −37 |
| 1 | 0.111721 | −1 | −0.5 | −3 | −3.4 | −9 | −3.4 | −4.2 |
| 2 | 0.135129 | −1 | −0.4 | −7 | 0.49 | 25 | −3.6 | 28.8 |
| 3 | 0.137064 | −1 | −0.8 | −7 | 0.54 | −47 | −1.3 | −13.8 |
| 4 | 0.0372113 | −1 | −0.3 | −2 | −0.34 | −22 | −2.8 | −19.8 |
| 6 | 0.101041 | −1 | −0.4 | −2 | −0.91 | −24 | −0.5 | −9.2 |
| 7 | 1.23142 | −1 | 0.0999999 | 14 | −0.0599995 | 4 | −0.599998 | 8.8 |
| 8 | 0.122128 | −1 | 0.3 | −9 | −1.15 | −33 | 2.6 | −27.2 |
| 9 | 0.590357 | −1 | 0.4 | 3 | 0.55 | 19 | −0.900002 | 5 |
| 11 | 0.142142 | −1 | 0.3 | −55 | 0.62 | 12 | −0.900002 | 14.8 |
| 13 | 0.0732453 | −1 | −0.4 | 2 | 0.47 | −21 | −1.1 | −8.60001 |
| 14 | 0.140047 | −1 | 0.8 | −6 | −0.87 | 39 | −2.2 | −13.8 |
| 15 | 0.0900951 | −1 | −0.2 | 14 | 1.67 | 23 | 0.800003 | 6.39999 |
| 16 | 0.368981 | −1 | 0.4 | 5 | −0.66 | 2 | 1.4 | 3.6 |
| 18 | 0.140893 | −1 | 0.5 | 4 | −1.3 | 93 | −1.1 | 46.6 |
| 19 | 0.25563 | −1 | −0.3 | 8 | −0.54 | 4 | −2.5 | 15.8 |
| 20 | 0.138076 | −1 | 0 | −7 | 0.14 | 36 | 1.6 | 66.4 |
| 21 | 0.146572 | −1 | 0.3 | 5 | −2.81 | 3 | −1.4 | 49 |
| 22 | 0.106868 | −1 | −0.0999999 | 0 | −0.8 | 6 | 0.400002 | 24.4 |
| 23 | 0.130652 | −1 | −0.2 | 1 | −0.23 | 37 | −0.400002 | 27.2 |
| 24 | 1.20573 | −1 | −0.2 | 1 | −3.83 | 17 | −2 | 18.6 |
| 25 | 0.138814 | −1 | 0.1 | −1 | 0.6 | 26 | −0.0999985 | 15.6 |
| 26 | 0.0740005 | −1 | 0 | −8 | 0.59 | 13 | −1.8 | 13.8 |
| 27 | 0.145416 | −1 | 0.3 | 1 | −2.41 | −6 | 0.300003 | 66.2 |
| 28 | 0.142654 | −1 | −0.3 | −1 | −6.33 | 120 | 1 | 99.2 |
| 29 | 0.147918 | −1 | 0 | 15 | 1.68 | 14 | −5.2 | 31.4 |
| 30 | 0.0163442 | −1 | 0.2 | 0 | −1.98 | −1 | 4 | −0.599998 |
| 31 | 0.138817 | −1 | 0.2 | 7 | −2.58 | 62 | 2 | −105.4 |
| 32 | 0.105927 | −1 | −0.4 | −6 | −1.47 | 5 | 2.8 | 9.2 |
| 36 | 0.00477362 | −1 | 0 | −2 | −1.19 | −1 | −0.300003 | −4.60001 |
| 38 | 0.117303 | −1 | −0.0999999 | −9 | 2.13 | 1 | −0.0999985 | 9 |
| 39 | 0.0178458 | −1 | 0.0999999 | 0 | −0.61 | −18 | −0.699997 | −18.4 |
| 40 | 0.118481 | −1 | 0.2 | −7 | 2.11 | −36 | −2 | −29.8 |
| 41 | 0.201572 | −1 | 0.6 | 1 | −3.8 | 24 | −0.699997 | 19.2 |
| 42 | 0.146081 | −1 | −0.3 | −19 | −1.78 | −4 | −2.3 | −22 |
| 43 | 0.134018 | −1 | 0.3 | −4 | −2.91 | 46 | 2 | 40.2 |
| 44 | 0.217328 | −1 | 0.0999999 | −1 | −1.47 | −33 | 3.2 | −33 |
| 45 | 0.13803 | −1 | −0.1 | 8 | −1.17 | −50 | 7 | −40.4 |
| 46 | 0.134785 | −1 | −0.5 | 4 | −3.57 | 31 | −0.699997 | 22.8 |
| 47 | 0.0279703 | −1 | 0.5 | 2 | −1.1 | 14 | −0.400002 | 9.4 |
| 48 | 0.0584051 | −1 | 0.8 | −8 | −2.59 | 11 | −0.5 | 5.4 |
| 49 | 0.116954 | −1 | 0 | 1 | 1.22 | 3 | −3.6 | −9.8 |
| 50 | 0.146858 | −1 | 0 | 0 | 2.61 | 128 | −1.3 | 19.6 |
| 51 | 0.131927 | −1 | 0.2 | −14 | −0.2 | 2 | 3.9 | 3.8 |
| 52 | 0.144709 | −1 | 0.2 | −1 | 1.45 | 18 | −1.7 | 12 |
| 53 | 0.139119 | −1 | −0.5 | −19 | −0.11 | 44 | 0.199997 | 20.8 |
| 54 | 0.0302882 | −1 | 0.5 | −2 | 0.25 | −23 | 0.599998 | −20 |
| 55 | 0.171913 | −1 | 0 | 1 | 2.79 | 3 | 0.0999985 | 0 |
| 56 | 0.106375 | −1 | 0.0999999 | −5 | 0.42 | −13 | 1.6 | −10.8 |
| 57 | 0.104169 | −1 | −0.0999999 | −3 | 0.95 | −12 | −1.8 | −20 |
| 58 | 0.0856091 | −1 | −0.5 | 3 | 1.98 | 26 | −1.9 | 4.4 |
| 59 | 0.0341288 | −1 | 0.4 | −1 | 0.98 | 2 | −4.8 | −5.6 |
| 60 | 0.097646 | −1 | 0 | 13 | 0.61 | 24 | 1.3 | 9.8 |
| 61 | 0.1395 | −1 | −0.3 | 9 | 0.31 | 53 | −4.9 | 25.6 |
| 63 | 0.68059 | −1 | 0 | 4 | 0.639999 | 18 | 0.599998 | 0 |
| 64 | 0.138946 | −1 | 0.2 | −1 | 0.0600004 | 61 | 0.300003 | 74.8 |
| 65 | 0.0532109 | −1 | 0.2 | 4 | −1.44 | −13 | −0.900002 | −11 |
| 67 | 0.0740617 | −1 | 0 | 1 | 1.04 | −3 | −3 | −7.8 |
| 68 | 0.0569683 | −1 | −0.5 | −2 | 1.4 | −9 | −0.900002 | −25 |
| 69 | 0.00691012 | −1 | 0.0999999 | −1 | −0.27 | −2 | −1.7 | −6.6 |
| 70 | 0.139186 | −1 | −0.6 | 1 | 1.48 | 47 | −5.2 | 25.2 |
| 71 | 0.126846 | −1 | 0 | 1 | 0.82 | 63 | −4.1 | 52.8 |
| 72 | 0.13637 | −1 | 0 | 2 | 1.64 | 35 | 0.699997 | 78.4 |
| 73 | 0.129615 | −1 | −0.2 | −7 | 1.06 | 17 | 0.400002 | 4.6 |
| 74 | 0.146678 | −1 | 0.0999999 | −16 | 0.63 | 23 | −0.900002 | −50.8 |
| 75 | 0.145033 | −1 | −0.5 | −11 | 1.89 | 27 | −2.5 | 11.6 |
| 76 | 0.0634667 | −1 | −0.3 | −2 | −0.0799999 | −33 | −0.300003 | −24.2 |
| 77 | 0.146049 | −1 | 0.4 | −26 | 0.9 | 65 | −1 | 42.8 |
| 78 | 0.0982358 | −1 | −0.7 | 5 | 0.81 | −28 | 0.599998 | −19.6 |
| 79 | 0.0684791 | −1 | 0.2 | 8 | 0.74 | 10 | 0.300003 | 2.79999 |
| 81 | 0.130661 | −1 | −0.4 | −2 | −0.76 | 51 | −1.3 | 54.2 |
| 82 | 0.394325 | −1 | −0.3 | −5 | 1.28 | 13 | 0.199997 | 2.8 |
| 84 | 0.139477 | −1 | −0.3 | −14 | −0.43 | −29 | −0.400002 | 2.8 |
| 86 | 0.100072 | −1 | 0 | 0 | −3.42 | 10 | 2.9 | 10.8 |
| 87 | 0.131969 | −1 | −0.0999999 | −4 | 4.44 | 15 | 0.699997 | −7.6 |
| 88 | 0.0872971 | −1 | −0.2 | −1 | 1.43 | −37 | −0.599998 | −22.8 |
| 89 | 0.147891 | −1 | −0.7 | 2 | 0 | 18 | −2.2 | 35.8 |
| 90 | 0.121465 | −1 | −0.0999999 | 0 | 2.14 | −21 | 2.1 | −24.2 |
| 91 | 0.11898 | −1 | 0.3 | −3 | −0.42 | 49 | −0.5 | 46.8 |
| 96 | 0.144155 | −1 | −0.6 | −17 | −0.64 | −64 | 3.9 | 63.4 |
| 97 | 0.351949 | −1 | −0.4 | 2 | 0.61 | 11 | −0.0999985 | 5.2 |
| 98 | 0.18757 | −1 | 0.0999999 | −5 | 0.860001 | 13 | 0 | −1.8 |
| 99 | 0.134868 | −1 | 0 | 22 | 0.52 | −6 | −4.5 | 7 |
| 100 | 0.117799 | −1 | 0.0999999 | 4 | 0.48 | −7 | 0.199997 | −25.2 |
| 101 | 0.0367795 | −1 | 0.1 | 3 | 3.06 | −10 | −1.2 | −3.59999 |
| 102 | 0.111677 | −1 | −0.3 | 4 | −1.38 | −28 | −1 | −24.6 |
| 103 | 0.114323 | −1 | 0.0999999 | 4 | −0.150001 | −25 | 2.2 | −12.2 |
| 104 | 0.133423 | −1 | 0.3 | −3 | 2.99 | −9 | −3.4 | −44.2 |
| 107 | 0.101947 | −1 | 0.5 | 1 | −3.77 | −8 | 7.3 | −3.8 |
| 108 | 0.116386 | −1 | 0.6 | −2 | 3.21 | 9 | 0.0999985 | 24.4 |
| 109 | 0.0974365 | −1 | 0.7 | 1 | 0.0599999 | −11 | 2.9 | −13.4 |
| 110 | 0.131864 | −1 | 0.0999999 | −14 | 1.95 | −5 | 1.9 | 0.800003 |
| 111 | 0.136136 | −1 | 0.5 | −9 | −2.76 | 36 | −0.5 | −6.6 |
| 112 | 0.114287 | −1 | 0.2 | −2 | −4.04 | −40 | −0.199997 | −25 |
| 113 | 0.138266 | −1 | −0.3 | −4 | −0.429999 | 42 | −0.5 | 16.8 |
| 114 | 0.125826 | −1 | 1 | 3 | 0.5 | −39 | 4.6 | −31.2 |
| 115 | 0.105199 | −1 | 0 | −4 | −2.45 | −15 | 1.6 | −21.6 |
| 116 | 0.0930752 | −1 | −0.2 | 6 | −3.22 | −9 | 4.9 | −3.4 |
| 117 | 0.128047 | −1 | 0.2 | 1 | −2.57 | −42 | −1.7 | −31.6 |
| 118 | 0.140806 | −1 | −0.3 | 0 | −0.0600004 | −62 | −0.599998 | −41.8 |
| 119 | 0.143808 | −1 | −0.3 | −23 | 4.33 | 6 | 0.5 | −38.4 |
| 120 | 0.0802399 | −1 | −0.2 | −4 | −1.02 | 7 | −2.9 | −2.40001 |
| 121 | 0.129812 | −1 | 0.5 | 3 | 0.61 | −11 | −0.300003 | −46.4 |
| 122 | 0.088658 | −1 | −0.2 | 4 | 1.21 | −10 | −1 | −9.8 |
| 123 | 0.0759396 | −1 | −0.0999999 | 0 | −0.77 | −13 | 3.4 | −2 |
| 124 | 0.13639 | −1 | 0.2 | 8 | −0.85 | 39 | −2.4 | 4 |
| 125 | 0.125841 | −1 | 0.5 | 2 | 0.179999 | −10 | 3.9 | 12.4 |
| 126 | 0.145163 | −1 | −0.4 | 7 | −2.91 | −72 | 3.6 | −45.6 |
| 127 | 0.0614093 | −1 | 0.1 | −1 | −0.25 | −13 | −0.300003 | −3.2 |
| 128 | 0.153188 | −1 | 0.1 | 3 | −3.1 | −2 | −0.699997 | −20.4 |
| 130 | 0.0650081 | −1 | −0.2 | 1 | −2.03 | 16 | −0.800003 | 16.6 |
| 131 | 0.137179 | −1 | 0.2 | −13 | −4.52 | 7 | 3 | 15.8 |
| 132 | 0.0864032 | −1 | 0.4 | −5 | −0.11 | −1 | 1.1 | 1.8 |
| 133 | 0.0929062 | −1 | −0.7 | 0 | 0.89 | −8 | 3.7 | 5.2 |
| 134 | 0.449477 | −1 | 0.3 | 5 | 1.42 | −6 | 1.4 | 3 |
| 135 | 0.0365772 | −1 | 0 | 1 | 0.9 | −17 | 2.1 | −10 |
| 137 | 0.0144675 | −1 | 0.4 | 7 | 2.1 | 14 | −1 | 6.40001 |
| 138 | 0.182175 | −1 | 0.5 | 3 | 3.5 | −3 | 1 | 11.6 |
| 139 | 0.0880844 | −1 | −0.8 | 2 | −4.7 | −18 | 0 | −14.2 |
| 140 | 0.111522 | −1 | −0.3 | 2 | 3.1 | −16 | −4 | −18.8 |
| 141 | 1.00253 | −1 | −0.4 | 3 | 1.9 | −3 | 1 | −0.399994 |
| 143 | 0.100334 | −1 | 0.3 | 0 | 4 | 3 | −2 | 12.6 |
| 144 | 0.0649467 | −1 | 0 | −2 | −3.2 | −18 | −1 | −14.4 |
| 145 | 0.146069 | −1 | −0.4 | −30 | 8.8 | 10 | −6 | 10.4 |
| 146 | 0.165746 | −1 | 0.4 | −8 | 1.2 | −13 | −1 | −4.40001 |
| 147 | 0.141038 | −1 | −0.2 | 24 | 1.8 | −5 | −1 | −0.400009 |
| 149 | 0.136185 | −1 | −0.4 | −4 | 1.8 | −55 | −1 | −10.4 |
| 150 | 0.148293 | −1 | 0.3 | −11 | −0.8 | −14 | −6 | 12.6 |
| 151 | 0.111327 | −1 | −0.2 | −2 | 0.4 | −31 | −1 | −18.6 |
| 152 | 0.0611758 | −1 | 0.2 | −3 | −1 | −23 | −2 | −20.4 |
| 153 | 0.14656 | −1 | 0 | −1 | 3.3 | −20 | −4 | −40.4 |
| 155 | 0.0378073 | −1 | −0.2 | 1 | 1 | 7 | −3 | −2 |
| 156 | 0.14594 | −1 | 0.5 | 5 | 8.5 | 100 | 1 | 95 |
| 157 | 0.143595 | −1 | 0.2 | 5 | 1.4 | 66 | −1 | 42.4 |
| 158 | 0.102347 | −1 | 1.1 | −1 | 2.6 | −16 | 4 | −5.6 |
| 159 | 0.131883 | −1 | −0.2 | −3 | 4.5 | 27 | 0 | 24 |
| 160 | 0.11986 | −1 | 0.4 | −6 | 1.9 | 5 | 2 | −6 |
| 161 | 0.139473 | −1 | 0.3 | −4 | 6.1 | 46 | −1 | 42.4 |
| 162 | 0.0358987 | −1 | 0 | 2 | 1.9 | −2 | −4 | 1.6 |
| 163 | 0.097877 | −1 | 0 | −6 | 1.5 | −6 | 0 | 2.4 |
| 164 | 0.0951882 | −1 | 0.2 | 5 | 2.2 | 10 | −2 | 11.8 |
| 165 | 0.702612 | −1 | 1.1 | 3 | 2.2 | 3 | 1 | 5.2 |
| 166 | 0.127836 | −1 | −0.5 | −6 | 1.7 | 34 | −3 | 24.4 |
| 167 | 0.175957 | −1 | −0.6 | 2 | 3.3 | 1 | −3 | 0.400009 |
| 168 | 0.146639 | −1 | −0.0999999 | −44 | −0.3 | 6 | −3 | 24 |
| 169 | 0.141726 | −1 | −0.2 | 11 | 3.3 | 28 | −3 | −1 |
| 170 | 0.0972743 | −1 | −0.2 | −2 | 3.1 | −8 | −5 | −27.6 |
| 171 | 0.133043 | −1 | −0.0999999 | 1 | 5.9 | 59 | −1 | 53 |
| 172 | 0.113641 | −1 | 0.2 | 0 | 5.3 | −13 | −3 | −26.6 |
| 173 | 0.281004 | −1 | 0.0999999 | −3 | 0.2 | 13 | −2 | 15.8 |
| 175 | 2.37902 | 1 | 0.9 | −1 | −0.0900002 | 16 | −0.5 | 2 |
| 176 | 1.86727 | 1 | −0.0999999 | −7 | 0.23 | −18 | 1.4 | 2.4 |
| 177 | 1.9921 | 1 | 0.8 | 2 | −0.02 | −32 | 1.1 | −39.8 |
| 178 | 1.86082 | 1 | −1 | −29 | 0.650001 | −12 | −2.8 | −9.4 |
| 179 | 2.42802 | 1 | 0.7 | 13 | −0.63 | 5 | 1.8 | 11.8 |
| 180 | 1.86444 | 1 | −1 | −10 | 3.91 | −20 | 1.3 | −28 |
| 181 | 1.85977 | 1 | 0.3 | −27 | 1.9 | −27 | −2 | −18.8 |
| 182 | 1.64609 | 1 | 0.4 | 5 | 3.7 | −1 | 2 | 2 |
| 183 | 1.47473 | 1 | −1 | 3 | 3.8 | −1 | 2 | 3.8 |
| 184 | 1.85531 | 1 | −0.3 | −31 | 1.2 | 4 | 3 | 3.59999 |
| 185 | 2.49548 | 1 | 0.4 | −1 | −0.8 | 18 | −3 | 19.8 |
| 186 | 1.86636 | 1 | 0.0999999 | −6 | 0.5 | −3 | −1 | −16.8 |

The separating surface of the foregoing may be represented as:

$${\sum }_{n = 1}^{k} {α}_{n} K ( x , {s}_{n} ) {y}_{n} + b = 0 ,$$

where,
k=162 is the number of support vectors,
αₙ is the Lagrange parameter for the n^{th} patient,
yₙ is the class label for the n^{th} patient,
b is the offset, and
K(x,sₙ) is the kernel function for the n^{th} patient defined as:
   K(x,sₙ)=e^{M}
in which:

$$M = {\sum }_{i = 1}^{6} {( {x}_{i} - d ( n , i ) )}^{2} / σ$$

where,
d(n,i),i=1,2, . . . ,6 are the values in columns 4 through 9.

Following are results obtained using the above trained and validated SVM as recorded at iterations of step 218. The confusion matrix is shown below as:

| | | PREDICTED CLASS | | |
|---|---|---|---|---|
| | | class 1 | class 2 | Accuracy |
| TRUE | class 1 | 175000 | 0 | 100% |
| CLASS | class 2 | 10162 | 1838 | 15.32% |

### Overall Accuracy 94.57%

Out of the 12,000 times the class 2 patients were tested, the SVM in this fourth example embodiment has correctly predicted them to be of class 2 on 1838 occasions. In this fourth SVM embodiment, there is 15.32 percent accuracy in predicting class 2 correctly. Additionally, the SVM of this fourth embodiment as described above accurately predicted all class 1 occurrences. Thus, there are no false positives indicated.

The foregoing describes embodiments and techniques used in connection with a machine learning predicting tool. In foregoing fourth embodiment, the six difference parameters: potassium, SGPT, glycosylated haemoglobin, cholesterol, chloride and LDL are used in connection with an SVM that may be used to predict which patients will develop diabetic nephropathy, as indicated by proteinuria, at time=6 months by examining test results at a time of 0 months and a subsequent set taken 3 months later. The times of 0 and 3 months are times relative to the 6 month time period being predicted.

It should be noted that the foregoing is not limited in applicability to diabetes mellitus and its complication diabetic nephropathy. The techniques described herein are applicable to any disease process and any of its complication. Additionally, specifics described in connection with the foregoing, such as time intervals of 3 months, should also not be construed as a limitation as other time intervals may be used in other embodiments in connection with other complications and diseases.

While the invention has been disclosed in connection with preferred embodiments shown and described in detail, their modifications and improvements thereon will become readily apparent to those skilled in the art. Accordingly, the spirit and scope of the present invention should be limited only by the following claims.

1. A method of disease prediction comprising:
using a machine learning tool to predict whether a member from a first class will belong to a second class after a predetermined amount of time, wherein members of said first class and said second class have a particular disease, and members of said first class do not have a particular complication after said predetermined amount of time and members of said second class do have said particular complication after said predetermined amount of time.

2. The method of claim 1, wherein said machine learning tool is used to predict who, among patients with diabetes mellitus, will develop proteinuria.

3. The method of claim 1, further comprising:
training said machine learning tool to minimize false positives wherein each of said false positives is defined as a number of patients incorrectly identified as developing proteinuria.

4. The method of claim 3, further comprising:
training said machine learning tool to maximize true positives wherein each of said true positives is defined as a number of patients correctly identified as developing proteinuria.

5. The method of claim 2, wherein said machine learning tool is a support vector machine, members of said first class have diabetes mellitus and do not have proteinuria after said predetermined amount of time, and members of said second class have diabetes mellitus and do have proteinuria after said predetermined amount of time.

6. The method of claim 5, further comprising:
predicting whether a member of said first class, given at least one input parameter at a first time period and three months later, will be a member of said second class six months from said first time period.

7. The method of claim 6, wherein at least one input parameter includes a value obtained using haemotology and blood biochemistry tests.

8. The method of claim 6, wherein said at least one input parameter is selected from the group consisting of: albumin, alkaline phosphates, SGOT, SGPT, calcium, cholesterol, chloride, creatinine kinase, creatinine, bicarbonate, iron, gamma GT, glucose, HDL cholesterol, potassium, lactate dehydrogenase, LDL, magnesium, sodium, phosphorus, total bilirubin, total protein, triglycerides, UIBC, urea, uric acid, glycosylated haemoglobin, white blood cells, differential counts, neutrophils, lymphocytes, monocytes, eosinophils, basophils, red blood corpuscles, hemoglobin, hematocrit, mean cell volume, mean cell hemoglobin, mean cell haemoglobin concentration, platelet count, erythrocyte sedimentation rate, reticulocyte count, peripheral smear, blood grouping, pH, specific gravity, glucose, protein, ketones, urobilinogen, ilirubin, nitrites, leukocytes, erythrocytes, epithelial cells, casts, and crystals.

9. The method of claim 8, wherein said at least one input parameter includes at least one difference parameter defined as a difference between a first value at said first time period and a second value three months later.

10. The method of claim 9, wherein said input parameters include potassium, SGPT, glycosylated haemoglobin, cholesterol, chloride and LDL.

11. The method of claim 9, wherein said input parameters are six difference parameters, each of said six difference parameters representing a difference between test values of one of six tests at a first time period and three months later, said six tests being potassium, SGPT, glycosylated haemoglobin, cholesterol, chloride and LDL.

12. The method of claim 5, wherein said support vector machine uses a Gaussian kernel function in defining a non-linear separating surface to separate members of said first class and said second class.

13. The method of claim 1, wherein said machine learning tool is used to predict who, among patients with diabetes mellitus, will develop diabetic nephropathy.

14. The method of claim 13, wherein at least one indicator is used to detect diabetic nephropathy, and the at least one indicator includes proteinuria.

15. The method of claim 6, further comprising:
partitioning an input data set into six partitions, each of said six partitions being approximately a same size and including an equal number of randomly selected members who belong to said second class at six months from said first time period and who are not in said second class at said first time period and three months later.

16. The method of claim 15, further comprising:
training said support vector machine with five of said six partitions; and
testing said support vector machine with said sixth partition.

17. A computer program product used for disease prediction comprising:
a machine learning tool that predicts whether a member from a first class will belong to a second class after a predetermined amount of time, wherein members of said first class and said second class have a particular disease, and members of said first class do not have a particular complication after said predetermined amount of time and members of said second class do have said particular complication after said predetermined amount of time.

18. The computer program product of claim 17, wherein said machine learning tool is used to predict who, among patients with diabetes mellitus, will develop proteinuria.

19. The computer program product of claim 17, further comprising:
machine executable code that trains said machine learning tool to minimize false positives wherein each of said false positives is defined as a number of patients incorrectly identified as developing proteinuria.

20. The computer program product of claim 19, further comprising:
machine executable code that trains said machine learning tool to maximize true positives wherein each of said true positives is defined as a number of patients correctly identified as developing proteinuria.

21. The computer program product of claim 18, wherein said machine learning tool is a support vector machine, members of said first class have diabetes mellitus and do not have proteinuria, and members of said second class have diabetes mellitus and do have proteinuria.

22. The computer program product of claim 21, further comprising:
machine executable code that predicts whether a member of said first class, given at least one input parameter at a first time period and three months later, will be a member of said second class six months from said first time period.

23. The computer program product of claim 22, wherein at least one input parameter includes a value obtained using haemotology and blood biochemistry tests.

24. The computer program product of claim 22, wherein said at least one input parameter is selected from the group consisting of: albumin, alkaline phosphates, SGOT, SGPT, calcium, cholesterol, chloride, creatinine kinase, creatinine, bicarbonate, iron, gamma GT, glucose, HDL cholesterol, potassium, lactate dehydrogenase, LDL, magnesium, sodium, phosphorus, total bilirubin, total protein, triglycerides, UIBC, urea, uric acid, glycosylated haemoglobin, white blood cells, differential counts, neutrophils, lymphocytes, monocytes, eosinophils, basophils, red blood corpuscles, hemoglobin, hematocrit, mean cell volume, mean cell hemoglobin, mean cell haemoglobin concentration, platelet count, erythrocyte sedimentation rate, reticulocyte count, peripheral smear, blood grouping, pH, specific gravity, glucose, protein, ketones, urobilinogen, ilirubin, nitrites, leukocytes, erythrocytes, epithelial cells, casts, and crystals.

25. The computer program product of claim 24, wherein said at least one input parameter includes at least one difference parameter defined as a difference between a first value at said first time period and a second value three months later.

26. The computer program product of claim 25, wherein said input parameters include potassium, SGPT, glycosylated haemoglobin, cholesterol, chloride and LDL.

27. The computer program product of claim 25, wherein said input parameters are six difference parameters, each of said six difference parameters representing a difference between test values of one of six tests at a first time period and three months later, said six tests being potassium, SGPT, glycosylated haemoglobin, cholesterol, chloride and LDL.

28. The computer program product of claim 21, wherein said support vector machine uses a Gaussian kernel function in defining a non-linear separating surface to separate members of said first class and said second class.

29. The computer program product of claim 17, wherein said machine learning tool is used to predict who, among patients with diabetes mellitus, will develop diabetic nephropathy.

30. The computer program product of claim 29, wherein at least one indicator is used to detect diabetic nephropathy, and the at least one indicator includes proteinuria.

31. The computer program product of claim 22, further comprising:
machine executable code that partitions an input data set into six partitions, each of said six partitions being approximately a same size and including an equal number of randomly selected members who belong to said second class at six months from said first time period and who are not in said second class at said first time period and three months later.

32. The computer program product of claim 31, further comprising:
machine executable code that trains said support vector machine with five of said six partitions; and
machine executable code that tests said support vector machine with said sixth partition.

33. A method of producing a support vector machine used in disease prediction comprising:
partitioning an input data set into a training data set and a testing data set, said input data set including members belonging to a first class and members belonging to a second class, wherein members of said first class and said second class have a particular disease, and members of said first class do not have a particular complication at a first time period and three and six months after said first time period and members of said second class have said particular complication at six months from said first time period, but not at said first time period and three months later.

34. The method of claim 33, further comprising:
training said machine support vector machine to minimize false positives wherein each of said false positives is defined as a number of patients incorrectly identified as developing proteinuria.

35. The method of claim 34, further comprising:
training said support vector machine to maximize true positives wherein each of said true positives is defined as a number of patients correctly identified as developing proteinuria.

36. The method of claim 35, wherein members of said first class have diabetes mellitus and do not have proteinuria, and members of said second class have diabetes mellitus and do have proteinuria at six months from said first time period.

37. The method of claim 36, wherein said input data set includes, for each member, at least one input parameter that is a value obtained from haemotology and blood biochemistry tests.

38. The method of claim 37, wherein said at least one input parameter is selected from the group consisting of: albumin, alkaline phosphates, SGOT, SGPT, calcium, cholesterol, chloride, creatinine kinase, creatinine, bicarbonate, iron, gamma GT, glucose, HDL cholesterol, potassium, lactate dehydrogenase, LDL, magnesium, sodium, phosphorus, total bilirubin, total protein, triglycerides, UIBC, urea, uric acid, glycosylated haemoglobin, white blood cells, differential counts, neutrophils, lymphocytes, monocytes, eosinophils, basophils, red blood corpuscles, hemoglobin, hematocrit, mean cell volume, mean cell hemoglobin, mean cell haemoglobin concentration, platelet count, erythrocyte sedimentation rate, reticulocyte count, peripheral smear, blood grouping, pH, specific gravity, glucose, protein, ketones, urobilinogen, ilirubin, nitrites, leukocytes, erythrocytes, epithelial cells, casts, and crystals.

39. The method of claim 38, wherein said at least one input parameter includes at least one difference parameter defined as a difference between a first value at said first time period and a second value three months later.

40. The method of claim 39, wherein said input parameters include potassium, SGPT, glycosylated haemoglobin, cholesterol, chloride and LDL.

41. The method of claim 39, wherein said input parameters are six difference parameters, each of said six difference parameters representing a difference between test values of one of six tests at a first time period and three months later, said six tests being potassium, SGPT, glycosylated haemoglobin, cholesterol, chloride and LDL.

42. The method of claim 33, wherein said support vector machine uses a Gaussian kernel function in defining a non-linear separating surface to separate members of said first class and said second class.

43. The method of claim 33, further comprising:
partitioning said input data set into six partitions, each of said six partitions being approximately a same size and including an equal number of randomly selected members who belong to said second class at six months from a first time period and who are not in said second class at said first time period and three months later;
training said support vector machine with five of said six partitions; and
testing said support vector machine with said sixth partition.

44. A computer program product that produces a support vector machine used in disease prediction comprising:
machine executable code that partitions an input data set into a training data set and a testing data set, said input data set including members belonging to a first class and members belonging to a second class, wherein members of said first class and said second class have a particular disease, and members of said first class do not have a particular complication at a first time period and three and six months after said first time period and members of said second class have said particular complication at six months from said first time period, but not at said first time period and three months later.

45. The computer program product of claim 44, further comprising:
machine executable code that trains said machine support vector machine to minimize false positives wherein each of said false positives is defined as a number of patients incorrectly identified as developing proteinuria.

46. The computer program product of claim 45, further comprising:
machine executable code that trains said support vector machine to maximize true positives wherein each of said true positives is defined as a number of patients correctly identified as developing proteinuria.

47. The computer program product of claim 46, wherein members of said first class have diabetes mellitus and do not have proteinuria, and members of said second class have diabetes mellitus and do have proteinuria at six months from said first time period.

48. The computer program product of claim 47, wherein said input data set includes, for each member, at least one input parameter that is a value obtained from haemotology and blood biochemistry tests.

49. The computer program product of claim 48, wherein said at least one input parameter is selected from the group consisting of: albumin, alkaline phosphates, SGOT, SGPT, calcium, cholesterol, chloride, creatinine kinase, creatinine, bicarbonate, iron, gamma GT, glucose, HDL cholesterol, potassium, lactate dehydrogenase, LDL, magnesium, sodium, phosphorus, total bilirubin, total protein, triglycerides, UIBC, urea, uric acid, glycosylated haemoglobin, white blood cells, differential counts, neutrophils, lymphocytes, monocytes, eosinophils, basophils, red blood corpuscles, hemoglobin, hematocrit, mean cell volume, mean cell hemoglobin, mean cell haemoglobin concentration, platelet count, erythrocyte sedimentation rate, reticulocyte count, peripheral smear, blood grouping, pH, specific gravity, glucose, protein, ketones, urobilinogen, ilirubin, nitrites, leukocytes, erythrocytes, epithelial cells, casts, and crystals.

50. The computer program product of claim 49, wherein said at least one input parameter includes at least one difference parameter defined as a difference between a first value at said first time period and a second value three months later.

51. The computer program product of claim 50, wherein said input parameters include potassium, SGPT, glycosylated haemoglobin, cholesterol, chloride and LDL.

52. The computer program product of claim 50, wherein said input parameters are six difference parameters, each of said six difference parameters representing a difference between test values of one of six tests at a first time period and three months later, said six tests being potassium, SGPT, glycosylated haemoglobin, cholesterol, chloride and LDL.

53. The computer program product of claim 44, wherein said support vector machine uses a Gaussian kernel function in defining a non-linear separating surface to separate members of said first class and said second class.

54. The computer program product of claim 44, further comprising:
machine executable code that partitions said input data set into six partitions, each of said six partitions being approximately a same size and including an equal number of randomly selected members who belong to said second class at six months from a first time period and who are not in said second class at said first time period and three months later;
machine executable code that trains said support vector machine with five of said six partitions; and
machine executable code that tests said support vector machine with said sixth partition.

55. A method of disease prediction comprising:
using a support vector machine to predict whether a member from a first class will belong to a second class after a predetermined amount of time, wherein members of said first class and said second class have diabetes mellitus, and members of said first class do not have proteinuria after said predetermined amount of time and members of said second class do have proteinuria after said predetermined amount of time, wherein input data of a patient used to predict whether the patient will belong to said first class or said second class includes input parameters based on test results including potassium, SGPT, glycosylated haemoglobin, cholesterol, chloride and LDL.

56. The method of claim 55, further comprising:
training said support vector machine to minimize false positives wherein each of said false positives is defined as a number of patients incorrectly identified as developing proteinuria.

57. The method of claim 56, further comprising:
training said support vector machine to maximize true positives wherein each of said true positives is defined as a number of patients correctly identified as developing proteinuria.

58. The method of claim 55, wherein said input data is based on test results of said patient at a first time period and three months later to predict whether the patient will develop proteinuria at six months from said first time period.

59. The method of claim 55, wherein said input data is based on test results of said patient at a first time period and three months later to predict whether the patient will develop diabetic nephropathy at six months from said first time period.

60. The method of claim 59, wherein said input parameters include at least one difference parameter defined as a difference between a first value of a test result at said first time period and a second value of said test result three months later.

61. The method of claim 60, wherein said input parameters are six difference parameters, each of said six difference parameters representing a difference between test values of one of six tests at a first time period and three months later, said six tests being potassium, SGPT, glycosylated haemoglobin, cholesterol, chloride and LDL.

62. The method of claim 61, wherein said support vector machine uses a Gaussian kernel function in defining a non-linear separating surface to separate members of said first class and said second class.

63. The method of claim 57, further comprising:
partitioning said input data set into six partitions, each of said six partitions being approximately a same size and including an equal number of randomly selected members who belong to said second class at six months from said first time period and who are not in said second class at said first time period and three months later.

64. A computer program product used for disease prediction comprising:
a support vector machine that predicts whether a member from a first class will belong to a second class after a predetermined amount of time, wherein members of said first class and said second class have diabetes mellitus, and members of said first class do not have proteinuria after said predetermined amount of time and members of said second class do have proteinuria after said predetermined amount of time, wherein input data of a patient used to predict whether the patient will belong to said first class or said second class includes input parameters based on test results including potassium, SGPT, glycosylated haemoglobin, cholesterol, chloride and LDL.

65. The computer program product of claim 64, further comprising:
machine executable code that trains said support vector machine to minimize false positives wherein each of said false positives is defined as a number of patients incorrectly identified as developing proteinuria.

66. The computer program product of claim 65, further comprising:
machine executable code that trains said support vector machine to maximize true positives wherein each of said true positives is defined as a number of patients correctly identified as developing proteinuria.

67. The computer program product of claim 64, wherein said input data is based on test results of said patient at a first time period and three months later to predict whether the patient will develop proteinuria at six months from said first time period.

68. The computer program product of claim 64, wherein said input data is based on test results of said patient at a first time period and three months later to predict whether the patient will develop diabetic nephropathy at six months from said first time period.

69. The computer program product of claim 68, wherein said input parameters include at least one difference parameter defined as a difference between a first value of a test result at said first time period and a second value of said test result three months later.

70. The computer program product of claim 69, wherein said input parameters are six difference parameters, each of said six difference parameters representing a difference between test values of one of six tests at a first time period and three months later, said six tests being potassium, SGPT, glycosylated haemoglobin, cholesterol, chloride and LDL.

71. The computer program product method of claim 70, wherein said support vector machine uses a Gaussian kernel function in defining a non-linear separating surface to separate members of said first class and said second class.

72. The computer program product of claim 66, further comprising:
machine executable code that partitions said input data set into six partitions, each of said six partitions being approximately a same size and including an equal number of randomly selected members who belong to said second class at six months from said first time period and who are not in said second class at said first time period and three months later.

73. The computer program product of claim 72, further comprising:
machine executable code that trains said support vector machine with five of said six partitions; and
machine executable code that tests said support vector machine with said sixth partition.

74. A computer-implemented method for disease prediction comprising:
predicting whether a member from a first class will belong to a second class after a predetermined amount of time, wherein members of said first class and said second class have diabetes mellitus, and members of said first class do not have proteinuria after said predetermined amount of time and members of said second class do have proteinuria after said predetermined amount of time, wherein said input data of a patient used to predict whether the patient will belong to said first class or said second class includes input parameters based on test results including potassium, SGPT, glycosylated haemoglobin, cholesterol, chloride and LDL.

75. The method of claim 74, wherein said input data includes at least one difference parameter that is a difference of a test result at a first time period and three months later.

76. The method of claim 75, further comprising:
using said input data of a patient to predict whether the patient will develop proteinuria after 6 months from said first time period.

77. A computer program product for disease prediction comprising:
machine executable code that predicts whether a member from a first class will belong to a second class after a predetermined amount of time, wherein members of said first class and said second class have diabetes mellitus, and members of said first class do not have proteinuria after said predetermined amount of time and members of said second class do have proteinuria after said predetermined amount of time, wherein said input data of a patient used to predict whether the patient will belong to said first class or said second class includes input parameters based on test results including potassium, SGPT, glycosylated haemoglobin, cholesterol, chloride and LDL.

78. The computer program product of claim 77, wherein said input data includes at least one difference parameter that is a difference of a test result at a first time period and three months later.

79. The computer program product of claim 77, further comprising:
machine executable code that uses said input data of a patient to predict whether the patient will develop proteinuria after 6 months from said first time period.

80. A computer-implemented method for producing a machine-learning tool used in disease prediction, the method comprising:
training said machine-learning tool using training data to predict whether a member from a first class will belong to a second class after a predetermined amount of time, wherein members of said first class and said second class have diabetes mellitus, and members of said first class do not have proteinuria after said predetermined amount of time and members of said second class do have proteinuria after said predetermined amount of time, wherein said training data includes, for each patient, input parameters based on test results including potassium, SGPT, glycosylated haemoglobin, cholesterol, chloride and LDL.

81. The method of claim 80, wherein said training data includes at least one difference parameter that is a difference of a test result at a first time period and three months later.

82. The method of claim 80, further comprising:
using input data of a patient to predict whether the patient will develop proteinuria after 6 months from said first time period.

83. A computer program product for producing a machine-learning tool used in
disease prediction, the computer program product comprising:
machine executable code that trains said machine-learning tool using training data to predict whether a member from a first class will belong to a second class after a predetermined amount of time, wherein members of said first class and said second class have diabetes mellitus, and members of said first class do not have proteinuria after said predetermined amount of time and members of said second class do have proteinuria after said predetermined amount of time, wherein said training data includes, for each patient, input parameters based on test results including potassium, SGPT, glycosylated haemoglobin, cholesterol, chloride and LDL.

84. The computer program product of claim 83, wherein said training data includes at least one difference parameter that is a difference of a test result at a first time period and three months later.

85. The method of claim 83, further comprising:
machine executable code that uses input data of a patient to predict whether the patient will develop proteinuria after 6 months from said first time period.
